SEQUENCE LISTING (66 sequences)

SEQ ID NO: 1 - protein, 100 aa, unidentified
  PEPTIDE 1..100: note = D1 domain of the human CD4 receptor
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGL

SEQ ID NO: 2 - protein, 78 aa, unidentified
  PEPTIDE 1..78: note = D2 domain of the human CD4 receptor
TANSDTHLLQGQSLTLTLESPPGSSPSVQCRSPRGKNIQGGKTLSVSQLELQDSGTWTCTVLQNQKKVEFKIDIVVLA

SEQ ID NO: 3 - protein, 232 aa, unidentified
  PEPTIDE 1..232: note = Fc portion of the human IgG1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 5 aa, synthetic construct
  PEPTIDE 1..5: note = Synthetic; Flexible linker sequence
GGGGS

SEQ ID NO: 5 - protein, 29 aa, unidentified
  PEPTIDE 1..29: note = N-terminal region of the human CCR5 receptor
MDYQVSSPIYDINYYTSEPCQKINVKQIA

SEQ ID NO: 6 - protein, 42 aa, synthetic construct
  PEPTIDE 1..42: note = Synthetic; M19 linker peptide
GGGGTAEAWYNLGNAYYKQGDYQKAIEYYQKALELDPNNAEA

SEQ ID NO: 7 - protein, 37 aa, synthetic construct
  PEPTIDE 1..37: note = Synthetic; M20 linker peptide
GGGGTAEAAAKEAAAKEAAAKEAAAKEAAAKEAAAKA

SEQ ID NO: 8 - protein, 50 aa, synthetic construct
  PEPTIDE 1..50: note = Synthetic; M21 linker peptide
GGGGTAGSAAGSGAGSAGSAAGSGAGSAGSAAGSGAGSAGSAAGSGAAGS

SEQ ID NO: 9 - protein, 61 aa, synthetic construct
  PEPTIDE 1..61: note = Synthetic; M22 linker peptide
GGGGTGAGAGGGGMFGSGGGGGGTGSTGPGYSFPHYGFPTYGGITFHPGTTKSNAGMKHGS

SEQ ID NO: 10 - protein, 36 aa, unidentified
  PEPTIDE 1..36: note = Human immunodeficiency virus type 1 T-20 polypeptide
YTSLIHSLIEESQNQQEKNEQELLELDKWASLWNWF

SEQ ID NO: 11 - protein, 34 aa, unidentified
  PEPTIDE 1..34: note = Human immunodeficiency virus type 1 C34 polypeptide
WMEWDREINNYTSLIHSLIEESQNQQEKNEQELL

SEQ ID NO: 12 - protein, 34 aa, unidentified
  PEPTIDE 1..34: note = Human immunodeficiency virus type 2 EHO polypeptide
WQQWERQVRFLDANITKLLEEAQIQQEKNMYELQ

SEQ ID NO: 13 - protein, 232 aa, unidentified
  PEPTIDE 1..232: note = Fc portion of the human IgG1 bearing the AM set of mutations(i.e. G236A, S239D, A330L and I332E point mutations)
EPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 232 aa, unidentified
  PEPTIDE 1..232: note = Fc portion of the human IgG1 bearing the A12 set of mutations(i.e. F243L, R292P and Y300L point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 232 aa, unidentified
  PEPTIDE 1..232: note = Fc portion of the human IgG1 bearing the A14 set of mutations(i.e. F243L, R292P and P396L point mutations)
EPKSCDKTHTCPPCPAPELLGGPSVFLLPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 16 - protein, 232 aa, unidentified
  PEPTIDE 1..232: note = Fc portion of the human IgG1 bearing the A16 set of mutations(i.e. F243L, R292P, Y300L and P396L point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 17 - protein, 232 aa, unidentified
  PEPTIDE 1..232: note = Fc portion of the human IgG1 bearing the A18 set of mutations(i.e. F243L, R292P, Y300L, P396L and V305I point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 18 - protein, 232 aa, unidentified
  PEPTIDE 1..232: note = Fc portion of the human IgG1 bearing the A41 set of mutations(i.e. S298A, E333A and K334A point mutations)
EPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 232 aa, unidentified
  PEPTIDE 1..232: note = Fc portion of the human IgG1 bearing the LL set of mutations(i.e. M428L and N434S point mutations)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - protein, 232 aa, unidentified
  PEPTIDE 1..232: note = Fc portion of the human IgG1 bearing the AM+LL set of mutations(i.e. G236A, S239D, A330L, I332E, M428L and N434S point mutations
EPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 21 - protein, 232 aa, unidentified
  PEPTIDE 1..232: note = Fc portion of the human IgG1 bearing the A12+LL set of mutations(i.e. F243L, R292P, Y300L, M428L and N434S point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 22 - protein, 232 aa, unidentified
  PEPTIDE 1..232: note = Fc portion of the human IgG1 bearing the A14+LL set of mutations(i.e. F243L, R292P, P396L, M428L and N434S point mutations)
EPKSCDKTHTCPPCPAPELLGGPSVFLLPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 23 - protein, 232 aa, unidentified
  PEPTIDE 1..232: note = Fc portion of the human IgG1 bearing the A16+LL set of mutations(i.e. F243L, R292P, Y300L, P396L, M428L and N434S pointmutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 24 - protein, 232 aa, unidentified
  PEPTIDE 1..232: note = Fc portion of the human IgG1 bearing the A18+LL set of mutations(i.e. F243L, R292P, Y300L, P396L, V305I, M428L and N434S pointmutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 25 - protein, 232 aa, unidentified
  PEPTIDE 1..232: note = Fc portion of the human IgG1 bearing the A41+LL set of mutations(i.e. S298A, E333A, K334A, M428L and N434S point mutations)
EPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 26 - protein, 232 aa, unidentified
  PEPTIDE 1..232: note = Fc portion of the human IgG1 bearing the A16_1 and LL sets ofmutations (i.e. S239D, F243L, R292P, Y300L, P396L, M428L andN434S point mutations)
EPKSCDKTHTCPPCPAPELLGGPDVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 27 - protein, 232 aa, unidentified
  PEPTIDE 1..232: note = Fc portion of the human IgG1 bearing the A16_2 and LL sets ofmutations (i.e. F243L, R292P, Y300L, K322A, P396L, M428L andN434S point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCAVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 28 - protein, 232 aa, unidentified
  PEPTIDE 1..232: note = Fc portion of the human IgG1 bearing the A16_3 and LL sets ofmutations (i.e. F243L, R292P, Y300L, I332E, P396L, M428L andN434S point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPEEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 29 - protein, 232 aa, unidentified
  PEPTIDE 1..232: note = Fc portion of the human IgG1 bearing the A16_4 and LL sets ofmutations (i.e. F243L, R292P, Y300L, K322A, I332E, P396L, M428Land N434S point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCAVSNKALPAPEEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 30 - protein, 232 aa, unidentified
  PEPTIDE 1..232: note = Fc portion of the human IgG1 bearing the A16_5 and LL sets ofmutations (i.e. F243L, R292P, Y300L, E333A, K334A, P396L, M428Land N434S point mutations)
EPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIAATISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 31 - protein, 232 aa, unidentified
  PEPTIDE 1..232: note = Fc portion of the human IgG1 bearing the A16_6 and LL sets ofmutations (i.e. S239D, F243L, R292P, Y300L, K322A, I332E, P396L,M428L and N434S point mutations)
EPKSCDKTHTCPPCPAPELLGGPDVFLLPPKPKDTLMISRTPEVTCVVVDVSHEDPEVKFNWYVDGVEVHNAKTKPPEEQYNSTLRVVSVLTVLHQDWLNGKEYKCAVSNKALPAPEEATISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 32 - protein, 232 aa, unidentified
  PEPTIDE 1..232: note = Fc portion of the human IgG1 bearing the A41_1 and LL sets ofmutations (i.e. S239D, F243L, R292P, S298A, E333A, K334A, M428Land N434S point mutations)
EPKSCDKTHTCPPCPAPELLGGPDVFLLPPKPKDTLMISRTPEVTCVVVDVSHEDPEVKFNWYVDGVEVHNAKTKPPEEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 33 - protein, 232 aa, unidentified
  PEPTIDE 1..232: note = Fc portion of the human IgG1 bearing the A41, A16 and LL sets ofmutations (i.e. F243L, R292P, S298A, E333A, K334A, M428L andN434S point mutations)
EPKSCDKTHTCPPCPAPELLGGPSVFLLPPKPKDTLMISRTPEVTCVVVDVSHEDPEVKFNWYVDGVEVHNAKTKPPEEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 34 - DNA, 300 bp, unidentified
  gene 1..300: note = D1 domain of the human CD4 receptor
  source 1..300: mol_type = genomic DNA
aagaaagtggtgctgggcaagaaaggcgacaccgtggaactgacctgcaccgccagccagaagaagtccatccagttccactggaagaacagcaaccagatcaagatcctgggcaaccagggcagcttcctgaccaagggccccagcaagctgaacgacagagccgactctcggcggagcctgtgggaccagggcaatttcccactgatcatcaagaacctgaagatcgaggacagcgacacctacatctgcgaggtggaagatcagaaagaagaggtgcagctgctggtgttcggcctg

SEQ ID NO: 35 - DNA, 234 bp, unidentified
  gene 1..234: note = D2 domain of the human CD4 receptor
  source 1..234: mol_type = genomic DNA
accgccaactccgacacccatctgctgcagggccagagcctgaccctgacactggaaagccctccaggcagcagccccagcgtgcagtgtagaagccccagaggcaagaacatccagggcggcaagaccctgagcgtgtcccagctggaactgcaggatagcggcacctggacctgtaccgtgctgcagaaccagaaaaaggtggaattcaagatcgacatcgtggtgctagcc

SEQ ID NO: 36 - DNA, 696 bp, unidentified
  gene 1..696: note = Fc portion of the human IgG1
  source 1..696: mol_type = genomic DNA
gagcccaagagctgcgacaagacccacacctgtcccccttgtcctgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtccctgagcctgagccctggaaaa

SEQ ID NO: 37 - DNA, 15 bp, synthetic construct
  gene 1..15: note = Synthetic; GGGGS flexible linker
  source 1..15: mol_type = other DNA
ggggggggaggctcc

SEQ ID NO: 38 - DNA, 87 bp, unidentified
  gene 1..87: note = N-terminal region of the human CCR5 receptor
  source 1..87: mol_type = genomic DNA
accgattatcaggtgtccagccccatctacgacatcaactactacaccagcgagccctgccagaaaatcaacgtgaagcagatcgcc

SEQ ID NO: 39 - DNA, 126 bp, synthetic construct
  gene 1..126: note = Synthetic; M19 linker peptide
  source 1..126: mol_type = other DNA
ggtggcggaggtaccgctgaggcctggtacaatctgggcaacgcctactacaagcagggcgactaccagaaggccatcgagtattatcagaaggcccttgagctggaccccaacaatgctgaagct

SEQ ID NO: 40 - DNA, 111 bp, synthetic construct
  gene 1..111: note = Synthetic; M20 linker peptide
  source 1..111: mol_type = other DNA
ggtggcggaggtaccgctgaagccgccgctaaagaagccgctgcaaaagaggctgccgccaaagaggcagctgctaaagaagcagcagccaaagaagccgccgcaaaggct

SEQ ID NO: 41 - DNA, 150 bp, synthetic construct
  gene 1..150: note = Synthetic; M21 linker peptide
  source 1..150: mol_type = other DNA
ggcggcggaggtaccgctggatctgctgctggaagcggagcaggctctgcaggttctgcagcaggatctggtgcaggttccgctggtagtgcagctggttctggcgctggctctgctggtagcgctgcaggcagcggagctgctggatcc

SEQ ID NO: 42 - DNA, 183 bp, synthetic construct
  gene 1..183: note = Synthetic; M22 linker peptide
  source 1..183: mol_type = other DNA
ggcggcggaggtaccggagctggtgctggtggcggaggaatgtttggatctggcggcggaggtggcggcacaggatctacaggacctggctacagcttccctcactacggctttccaacctacggcggcatcacatttcaccccggcaccaccaagtctaacgccggcatgaagcacggatcc

SEQ ID NO: 43 - DNA, 108 bp, unidentified
  gene 1..108: note = Human immunodeficiency virus type 1 T-20 polypeptide
  source 1..108: mol_type = genomic DNA
tacacaagcctgatccacagcctgatcgaggaaagccagaaccagcaggaaaagaacgagcaggaactgctggaactggacaagtgggccatcctgtggaattggttt

SEQ ID NO: 44 - DNA, 102 bp, unidentified
  gene 1..102: note = Human immunodeficiency virus type 1 C34 polypeptide
  source 1..102: mol_type = genomic DNA
tggatggaaatggacagagagatcaacaattacaccagcctgatccactccctgatcgaggaaagccagaaccagcaggaaaagaacgagcaggaactgctg

SEQ ID NO: 45 - DNA, 102 bp, unidentified
  gene 1..102: note = Human immunodeficiency virus type 2 EHO polypeptide
  source 1..102: mol_type = genomic DNA
tggcagcagtgggaaagacaagtgcggttcctggacgccaacatcaccaagctgctggaagaggcccagatccagcaagagaagaatatgtacgagctgcag

SEQ ID NO: 46 - DNA, 696 bp, unidentified
  gene 1..696: note = Fc portion of the human IgG1 bearing the AM set of mutations(i.e. G236A, S239D, A330L and I332E point mutations)
  source 1..696: mol_type = genomic DNA
gagcccaagagctgcgacaagacccacacctgtcccccttgtcctgcccctgaactgctggccggacccgacgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtgtccaacaaggccctgcctctgcccgaggaaaagaccatcagcaaggccaagggccagcccagggaaccccaggtgtacacactgccccccagcagagatgagctgaccaagaaccaggtgtccctgacctgtctcgtgaagggcttttacccctccgatatcgccgtggaatgggagagcaacggccagcccgagaacaactacaagaccaccccccctgtgctggactccgacggctcattcttcctgtactccaagctgaccgtggacaagagcagatggcagcagggcaacgtgttcagctgctccgtgatgcacgaggccctgcacaaccactacacccagaagtccctgagcctgagcccaggcaaa

SEQ ID NO: 47 - DNA, 696 bp, unidentified
  gene 1..696: note = Fc portion of the human IgG1 bearing the A12 set of mutations(i.e. F243L, R292P and Y300L point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtccctgagcctgagccctggaaaa

SEQ ID NO: 48 - DNA, 696 bp, unidentified
  gene 1..696: note = Fc portion of the human IgG1 bearing the A14 set of mutations(i.e. F243L, R292P and P396L point mutations)
  source 1..696: mol_type = genomic DNA
gagcccaagagctgcgacaagacccacacctgtcccccttgtcctgcccctgaactgctggccggacccgacgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtgtccaacaaggccctgcctctgcccgaggaaaagaccatctccaaggccaagggccagcccagggaaccccaggtgtacacactgccccccagcagagatgagctgaccaagaaccaggtgtccctgacctgtctcgtgaagggcttttacccctccgatatcgccgtggaatgggagagcaacggccagcccgagaacaactacaagaccaccccccctgtgctggactccgacggctcattcttcctgtactccaagctgaccgtggacaagagccggtggcagcagggcaacgtgttcagctgcagcgtgatgcacgaggccctgcacaaccactacacccagaagtccctgagcctgagcccaggcaaa

SEQ ID NO: 49 - DNA, 696 bp, unidentified
  gene 1..696: note = Fc portion of the human IgG1 bearing the A16 set of mutations(i.e. F243L, R292P, Y300L and P396L point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgatgcacgaggccctgcacaaccactacacccagaagtccctgagcctgagccctggaaaa

SEQ ID NO: 50 - DNA, 696 bp, unidentified
  gene 1..696: note = Fc portion of the human IgG1 bearing the A18 set of mutations(i.e. F243L, R292P, Y300L, P396L and V305I point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccatcctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgatgcacgaggccctgcacaaccactacacccagaagtccctgagcctgagccctggaaaa

SEQ ID NO: 51 - DNA, 696 bp, unidentified
  gene 1..696: note = Fc portion of the human IgG1 bearing the A41 set of mutations(i.e. S298A, E333A and K334A point mutations)
  source 1..696: mol_type = genomic DNA
gagcccaagagctgcgacaagacccacacctgtcccccttgtcctgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacgccacctaccgggtggtgtccgtgctgaccgtgctgcaccaggactggctgaacggcaaagagtacaagtgcaaggtgtccaacaaggccctgcctgcccccatcgccgccaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgatgcacgaggccctgcacaaccactacacccagaagtccctgagcctgagccctggaaaa

SEQ ID NO: 52 - DNA, 696 bp, unidentified
  gene 1..696: note = Fc portion of the human IgG1 bearing the LL set of mutations(i.e. M428L and N434S point mutations)
  source 1..696: mol_type = genomic DNA
gagcccaagagctgcgacaagacccacacctgtcccccttgtcctgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 53 - DNA, 696 bp, unidentified
  gene 1..696: note = Fc portion of the human IgG1 bearing the AM+LL set of mutations(i.e. G236A, S239D, A330L, I332E, M428L and N434S pointmutations)
  source 1..696: mol_type = genomic DNA
gagcccaagagctgcgacaagacccacacctgtcccccttgtcctgcccctgaactgctggccggacccgacgtgttcctgttccccccaaagcccaaggacaccctgatgatctcccggacccccgaagtgacctgcgtggtggtggatgtgtcccacgaggaccctgaagtgaagttcaattggtacgtggacggcgtggaagtgcacaacgccaagaccaagcccagagaggaacagtacaacagcacctaccgggtggtgtccgtgctgacagtgctgcaccaggactggctgaacggcaaagagtacaagtgcaaggtgtccaacaaggccctgcctctgcccgaggaaaagaccatctccaaggccaagggccagcccagggaaccccaggtgtacacactgccccccagcagagatgagctgaccaagaaccaggtgtccctgacctgtctcgtgaagggcttttacccctccgatatcgccgtggaatgggagagcaacggccagcccgagaacaactacaagaccaccccccctgtgctggactccgacggctcattcttcctgtactccaagctgaccgtggacaagagccggtggcagcagggcaacgtgttcagctgcagcgtgctgcacgaggccctgcactcccactacacccagaagtccctgagcctgagcccaggcaaa

SEQ ID NO: 54 - DNA, 696 bp, unidentified
  gene 1..696: note = Fc portion of the human IgG1 bearing the A12+LL set of mutations(i.e. F243L, R292P, Y300L, M428L and N434S point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 55 - DNA, 696 bp, unidentified; identical to SEQ ID NO: 53
  gene 1..696: note = Fc portion of the human IgG1 bearing the A14+LL set of mutations(i.e. F243L, R292P, P396L, M428L and N434S point mutations)
  source 1..696: mol_type = genomic DNA

SEQ ID NO: 56 - DNA, 696 bp, unidentified
  gene 1..696: note = Fc portion of the human IgG1 bearing the A16+LL set of mutations(i.e. F243L, R292P, Y300L, P396L, M428L and N434S pointmutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 57 - DNA, 696 bp, unidentified
  gene 1..696: note = Fc portion of the human IgG1 bearing the A18+LL set of mutations(i.e. F243L, R292P, Y300L, P396L, V305I, M428L and N434S pointmutations
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccatcctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 58 - DNA, 696 bp, unidentified
  gene 1..696: note = Fc portion of the human IgG1 bearing the A41+LL set of mutations(i.e. S298A, E333A, K334A, M428L and N434S point mutations)
  source 1..696: mol_type = genomic DNA
gagcccaagagctgcgacaagacccacacctgtcccccttgtcctgcccctgaactgctgggcggacctagcgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagcccagagaggaacagtacaacgccacctaccgggtggtgtccgtgctgaccgtgctgcaccaggactggctgaacggcaaagagtacaagtgcaaggtgtccaacaaggccctgcctgcccccatcgccgccaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 59 - DNA, 696 bp, unidentified
  gene 1..696: note = Fc portion of the human IgG1 bearing the A16_1 and LL sets ofmutations (i.e. S239D, F243L, R292P, Y300L, P396L, M428L andN434S point mutations)
  source 1..696: mol_type = genomic DNA
gagcccaagagctgcgacaagacccacacctgtcccccttgtcctgcccctgaactgctgggcggacctgacgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 60 - DNA, 696 bp, unidentified
  gene 1..696: note = Fc portion of the human IgG1 bearing the A16_2 and LL sets ofmutations (i.e. F243L, R292P, Y300L, K322A, P396L, M428L andN434S point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcgcagtgtccaacaaggccctgcctgcccccatcgagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 61 - DNA, 696 bp, unidentified
  gene 1..696: note = Fc portion of the human IgG1 bearing the A16_3 and LL sets ofmutations (i.e. F243L, R292P, Y300L, I332E, P396L, M428L andN434S point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccgaggagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 62 - DNA, 696 bp, unidentified
  gene 1..696: note = Fc portion of the human IgG1 bearing the A16_4 and LL sets ofmutations (i.e. F243L, R292P, Y300L, K322A, I332E, P396L, M428Land N434S point mutations)
  source 1..696: mol_type = genomic DNA
gagcctaagagctgcgacaagacccacacctgtcctccatgtcctgctccagaactgctcggcggaccttccgtttttctgctgcctcctaagcctaaggacaccctgatgatctccagaacacccgaagtgacctgcgtggtggtggatgtgtcccacgaagatcctgaagtgaagttcaattggtacgtggacggcgtggaagtgcacaacgccaagacaaagcctcctgaggaacagtacaacagcaccctgagagtggtgtccgtgctgacagtgctgcaccaggattggctgaacggcaaagagtacaagtgcgccgtgtccaacaaggccctgccagctccagaggaaaagaccatctccaaggccaagggccagcctagagaaccccaggtgtacacactgcctccaagcagagatgagctgaccaagaaccaggtgtccctgacctgtctggtcaagggcttctacccctccgatatcgccgtggaatgggagagcaatggacagcccgagaacaactacaagacaacccctctggtgctggactccgacggctcattcttcctgtactccaagctgaccgtggacaagagcagatggcagcagggcaacgtgttcagctgctctgtgctgcacgaagccctgcacagccactacacccagaagtccctgtctctgagccctggaaaa

SEQ ID NO: 63 - DNA, 696 bp, unidentified
  gene 1..696: note = Fc portion of the human IgG1 bearing the A16_5 and LL sets ofmutations (i.e. F243L, R292P, Y300L, E333A, K334A, P396L, M428Land N434S point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcaaggtgtccaacaaggccctgcctgcccccatcgccgccaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagcccaggcaaa

SEQ ID NO: 64 - DNA, 696 bp, unidentified
  gene 1..696: note = Fc portion of the human IgG1 bearing the A16_6 and LL sets ofmutations (i.e. S239D, F243L, R292P, Y300L, K322A, I332E, P396L,M428L and N434S point mutations)
  source 1..696: mol_type = genomic DNA
gagcccaagagctgcgacaagacccacacctgtcccccttgtcctgcccctgaactgctgggcggacctgacgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacagcaccctgcgggtggtgtccgtgctgaccgtgctgcaccaggactggctgaacggcaaagagtacaagtgcgcagtgtccaacaaggccctgcctgcccccgaggagaaaaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccacccccctggtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagccctggaaaa

SEQ ID NO: 65 - DNA, 696 bp, unidentified
  gene 1..696: note = Fc portion of the human IgG1 bearing the A41_1 and LL sets ofmutations (i.e. S239D, F243L, R292P, S298A, E333A, K334A, M428Land N434S point mutations)
  source 1..696: mol_type = genomic DNA
gagcccaagagctgcgacaagacccacacctgtcccccttgtcctgcccctgaactgctgggcggacctagcgtgttcctgctgcccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagaccaagccccctgaggaacagtacaacgccacctaccgggtggtgtccgtgctgaccgtgctgcaccaggactggctgaacggcaaagagtacaagtgcaaggtgtccaacaaggccctgcctgcccccatcgccgccaccatcagcaaggccaagggccagccccgcgaaccccaggtgtacacactgccccctagcagggacgagctgaccaagaaccaggtgtccctgacctgtctcgtgaagggcttctacccctccgatatcgccgtggaatgggagagcaacggccagcccgagaacaactacaagaccaccccccctgtgctggacagcgacggctcattcttcctgtacagcaagctgacagtggacaagagccggtggcagcagggcaacgtgttcagctgcagcgtgctgcacgaggccctgcactcccactacacccagaagtccctgagcctgagccctggaaaa

SEQ ID NO: 66 - DNA, 696 bp, unidentified; identical to SEQ ID NO: 65
  gene 1..696: note = Fc portion of the human IgG1 bearing the A41, A16 and LL sets ofmutations (i.e. F243L, R292P, S298A, E333A, K334A, M428L andN434S point mutations)
  source 1..696: mol_type = genomic DNA

(19) United States
(12) Patent Application Publication
(10) Pub. No.: US 2023/0014906 A1
(43) Pub. Date: Jan. 19, 2023

(54) FC-FUSION PROTEIN DERIVATIVES WITH HIGH DUAL HIV ANTIVIRAL AND IMMUNOMODULATORY ACTIVITY

(71) Applicant: ALBAJUNA THERAPEUTICS, S.L., BADALONA (BARCELONA) (ES)

(72) Inventors: JORGE CARRILLO MOLINA, BADALONA (BARCELONA) (ES); BONAVENTURA CLOTET SALA, BADALONA (BARCELONA) (ES); JULIA M. BLANCO ARBUES, BADALONA (BARCELONA) (ES)

(21) Appl. No.: 17/816,708

(22) Filed: Aug. 1, 2022

Related U.S. Application Data
(63) Continuation of application No. 16/612,727, filed on Nov. 11, 2019.
(60) Provisional application No. 62/504,411, filed on May 10, 2017.

(51) Int. Cl. C07K 14/735; C07K 14/16; C07K 14/73; C12N 15/62; C07K 14/005; C07K 14/72

(52) U.S. Cl. CPC C07K 14/70535; C07K 14/162; C07K 14/70514; C12N 15/62; C07K 14/005; C07K 14/723; A61P 31/18

(57) ABSTRACT

Fc-fusion protein derivatives against HIV have enhanced yield in mammalian cells, and extended antiviral and immunomodulatory activities. The Fc-fusion protein derivatives can block the entry of human immunodeficiency virus (HIV) into host cells, elicit effector functions through the activation of natural killer (NK) and other immune system cells, can be produced with high yield in mammalian cells, and have extended activity in vivo. Nucleic acids, vectors and host cells can express the Fc-fusion protein derivatives, which have therapeutic and diagnostic applications in human health.

### PRIORITY AND CROSS REFERENCE TO RELATED APPLICATIONS

This application is a Continuation Application of U.S. application Ser. No. 16/612,727, filed Nov. 11, 2019, which is the U.S. National Stage Application under 35 U.S.C. § 371 of International Application No. PCT/IB2018/000602, filed May 9, 2018, designating the U.S. and published in English as WO 2018/207023 A2 on Nov. 15, 2018, which claims the benefit of U.S. Provisional Application No. U.S. 62/504,411, filed May 10, 2017. Any and all applications for which a foreign or a domestic priority is claimed is/are identified in the Application Data Sheet filed herewith and is/are hereby incorporated by reference in their entirety under 37 C.F.R. § 1.57.

### COPYRIGHT STATEMENT

A portion of the disclosure of this patent document contains material which is subject to (copyright or mask work) protection. The (copyright or mask work) owner has no objection to the facsimile reproduction by anyone of the patent document or the patent disclosure, as it appears in the Patent and Trademark Office patent file or records, but otherwise reserves all (copyright or mask work) rights whatsoever.

### SEQUENCE LISTING IN ELECTRONIC FORMAT

The present application is being filed along with an Electronic Sequence Listing as an xml file via EFS-Web. The Electronic Sequence Listing is provided as a file entitled DURC075001APCSEQLIST.xml, created and last saved on Jul. 28, 2022, which is 109,505 bytes in size. The information in the Electronic Sequence Listing is incorporated herein by reference in its entirety.

### FIELD

The present invention relates to Fc-fusion protein derivatives against HIV with enhanced yield in mammalian cells, extended antiviral and immunomodulatory activities. The Fc-fusion protein derivatives of the present invention are characterized for: (i) blocking the entry of human immunodeficiency virus (HIV) into host cells, (ii) eliciting selective effector functions through the activation of natural killer (NK) and other immune system cells, (iii) having a high yield production in mammalian cells and (iv) having extended activity in vivo. Various forms of these polypeptides are disclosed and exemplified. Isolated nucleic acids, vectors and host cells expressing these polypeptides, as well their therapeutic and diagnostic applications in human health, are also within the scope of the present invention.

### BACKGROUND

HIV infection is one of the major threats to global human health. It is estimated that more than 78 million people worldwide have been infected by the human immunodeficiency virus since 1981. Nearly half of these infected individuals have died of the resultant Acquired Immunodeficiency Syndrome (AIDS) during the same time frame. See UNAIDS, unaids.org/, October 2015.

The generation of protective antibodies is the main mechanism for developing vaccines against human pathogens. However, the development of immunogens able to elicit such antibodies against HIV has failed so far. The design of these immunogens requires, firstly, identifying conserved epitopes to assure a continuous and stable response and, secondly, devising new and more efficient immunogens that present those epitopes properly. See Haynes B, Curr Opin Immunol. 2015; 35:39-47. In contrast to these insufficient advances in immunogen design, a large number of new, potent and broad-spectrum antibodies (i.e. broadly neutralizing antibodies, bNAbs) against HIV envelope glycoprotein isolated from HIV infected individuals have been identified recently. See Mascola J, et al., Immunol Rev. 2013; 254:225-244. In addition, synthetic molecules based on antibody structure have been also proposed as new therapeutic agents. See Gardner M, et al., Nature 2015; 519:87-91. Indeed, highly potent antibodies may protect uninfected individuals from HIV acquisition or may be used in eradication strategies in HIV infected patients. See Mascola, 2013, supra. The use of neutralizing antibodies against the envelope glycoprotein and its subunits has also been proposed to prevent HIV replication in vivo. See Yang X, et al., J. Virol. 2005; 79:3500-3508.

In therapeutic grounds, antibodies are especially relevant due to their dual function as antiviral agents able to block HIV replication through binding to HIV envelope glycoprotein and as NK cell activators though the interaction of constant regions of antibody chains (Fc) with the Fc receptor CD16 expressed on the surface of NK and other cells. This interaction enables CD16+ immune cells to kill infected cells through a mechanism known as antibody-dependent cellular cytotoxicity (ADCC). See Milligan C, et al., Cell Host Microbe. 2015; 17:500-506. Since both activities seem to be required for the protection of uninfected subjects, significant efforts have been made to develop neutralizing antibodies with increased antiviral and ADCC activities. Several IgG derivatives have been described that increase the affinity of human IgG1 for human CD16, therefore increasing their ADCC activity. See Saxena A, et al., Frontiers Immunol 2016; 7(580):1-11. However, the clinical application of these modified Fc-fusion proteins can be affected by their low level of production in mammalian cells or their changes in effector functions. Therefore, there is a need in the art for neutralizing antibodies with increased ADCC activity that possess receptor selectivity and a high level of production.

### SUMMARY

In a first aspect, the present application is directed to the modified Fc-fusion protein derivatives of the invention which comprise from the N- to C-terminus:
(a) the D1 and D2 extracellular domains of a human CD4 receptor,
(b) the Fc portion of a human IgG1 comprising at least one of a M428L or N434S point mutations,
(c) a moiety selected from the group consisting of
   (i) a linker polypeptide of sequence (GGGGS)ₙ wherein 1≤n≤10 (GGGGS=SEQ ID NO: 4),
   (ii) SEQ ID NO:5-9, and
   (iii) combinations thereof, and
(d) a gp41-derived polypeptide.

The Fc-fusion protein derivatives of the invention have an antiviral and ADCC activity higher than any other comparable antibodies known in the art. See Gardner, 2015, supra. The expression and duration of action of the Fc-fusion protein derivatives of the invention are also higher than other comparable anti-HIV antibodies.

In an additional aspect, the invention relates to the nucleic acids encoding the Fc-fusion protein derivatives of the invention, to vectors comprising said nucleic acids and to host cells comprising the nucleic acids and vectors indicated before.

In a further aspect, the invention refers to pharmaceutical compositions comprising the Fc-fusion protein derivatives, nucleic acids, vectors and host cells of the invention, or mixtures thereof.

In another aspect, the invention is directed to a combination comprising the Fc-fusion protein derivatives, nucleic acids, vectors, host cells and pharmaceutical compositions of the invention and at least one therapeutic agent.

In a still further aspect, the invention relates to the use of the Fc-fusion protein derivatives, nucleic acids, vectors, host cells, pharmaceutical compositions and combinations of the invention, or mixtures thereof, as a medicament. In a further version of this aspect, the invention refers to the use of the Fc-fusion protein derivatives, nucleic acids, vectors, host cells, pharmaceutical compositions and combinations of the invention, or mixtures thereof, in the treatment or prevention of HIV infection or AIDS. In an alternative form of this aspect, the invention relates to a method of treating or preventing HIV infection or AIDS in a subject which comprises the administration of a therapeutically effective amount of the Fc-fusion protein derivatives, nucleic acids, vectors, host cells, pharmaceutical compositions and combinations of the invention, or mixtures thereof, to the subject. In a further alternative form of this aspect, the invention refers to the use of the Fc-fusion protein derivatives, nucleic acids, vectors, host cells, pharmaceutical compositions and combinations of the invention, or mixtures thereof, in the manufacture of a medicament for the treatment or prevention of HIV infection or AIDS.

Additionally, the present invention relates to a method of preparing the Fc-fusion protein derivatives of the invention which comprises the steps of (a) culturing a host cell comprising a nucleic acid according to the invention, (b) expressing the nucleic acid sequence and (c) recovering the Fc-fusion protein derivative from the host cell culture.

In another aspect, the invention refers to a method of inactivating HIV which comprises the step of contacting the virus with a Fc-fusion protein derivative of the invention.

In an additional aspect, the invention is directed to a method of inducing the expression of gp120 in a HIV infected cell which comprises the step of contacting the infected cell with the Fc-fusion protein derivatives, nucleic acids, vectors, host cells, pharmaceutical compositions and combinations of the invention, or a mixture thereof.

In a further aspect, the invention relates to a method of detecting HIV in a sample which comprises the steps of (a) contacting the sample with a Fc-fusion protein derivative of the invention and (b) determining whether the Fc-fusion protein derivative specifically binds to a molecule of the sample.

In a still further aspect, the invention relates to a kit comprising the Fc-fusion protein derivatives, nucleic acids, vectors, host cells and pharmaceutical compositions of the invention, or mixtures thereof.

### BRIEF DESCRIPTION OF THE DRAWINGS

FIG. 1. Schematic representation of the general structure of the Fc-fusion protein derivatives of the invention. From N- to C-terminus the molecules contain the D1 and D2 domains of human CD4, the constant region of human IgG1 (e.g. wild-type, mutated), optionally a human CCR5 peptide, a flexible linker and a HIV gp41 peptide (e.g. T-20, C34 or EHO).

FIG. 2. Schematic view of screening procedures carried out to improve activity and yield of Fc-fusion proteins. Different sets of mutations have been screened in the human IgG1 sequence to improve production and effector functions. See Table 1. Different linkers and the gp41-derived polypeptides T20, C34 and EHO were then screened.

FIGS. 3A-3C. Production kinetics of various Fc-fusion protein derivatives of the invention. All IgG1 mutations have been introduced to the M7 IgG1 wt C34 molecule.

FIGS. 4A-4C. Neutralizing activity of several Fc-fusion protein derivatives of the invention. All mutations have been introduced to the M7 IgG1 wt C34 molecule. FIG. 4A) The figure depicts IC₅₀ values for the following HIV isolates: NL4.3, BaL, AC10 and SVPB16. The geometric means of the IC₅₀ values for each Fc-fusion protein derivative are shown. FIG. 4B) The figure shows the effect of LL mutations on neutralizing activity against the isolate BaL. FIG. 4C) The figure shows the effect of different combinations of mutations on neutralizing activity against the isolate BaL.

FIGS. 5A-5C. ADCC activity of the different mutated IgG fusion proteins of the invention. EC50 values for ADCC were normalized to the reference molecule Ml. Lower values indicate higher activity. FIG. 5A) ADCC activity of A12 to A141 series. FIG. 5B) The figure shows the effect of LL mutations on ADCC activity. FIG. 5C) The Figure shows the effect of different combinations of mutations on ADCC activity.

FIG. 6. Binding to CD32a, CD32bc, Cd16a, CD16aVF, CD64 and C1q of the different mutated IgG fusion proteins of the invention. Fold changes in ELISA binding signals were calculated for each compound using the wt IgG1 molecule M7AC34 (not shown). Fold changes were Log2 transformed and clustered.

FIGS. 7A-7C. Binding affinity of several Fc-fusion protein derivatives of the invention having extended activity to a recombinant human neonatal Fc Receptor (FcRn). ELISA assays were performed at pH=6.0 (upper plots) and pH=7.2 (lower plots).

FIGS. 8A-8C. Effect of different linkers on production, neutralization and ADCC activity of the fusion proteins of the invention. FIG. 8A) Production was assessed in 293T cells until day seven post-transfection. FIG. 8B) neutralizing activity was assayed against to HIV-1 isolates BaL and AC10. FIG. 8C) ADCC activity is shown as in FIGS. 5A-5C.

FIGS. 9A-9C. Effect of different gp41 peptides on production, neutralization and ADCC activity of the fusion proteins of the invention. FIG. 9A) Production was assessed in 293T cells until day seven post-transfection. FIG. 9B) neutralizing activity was assayed against the HIV-1 isolate BaL. FIG. 9C) ADCC activity is shown as raw dose-response data (M7BC34 molecule, an IgG2 derivative, is used as negative control).

FIGS. 10A-10B. In vivo effect of the fusion proteins of the invention. FIG. 10A) Schematic representation of in vivo experiments. FIG. 10B) Analysis of infected cells (HIV GAG+ cells) after two weeks of infection in the spleen of untreated (CTROL) and treated animals (M20A16_4 LLC34 and M21A16_4 LLC34).

FIG. 11. Diagram of the expression plasmid pM5A16T20. The main characteristics of the plasmid, such as selectable marker and open reading frames, are shown.

FIG. 12. Diagram of the expression plasmid pM7A16LLC34. The main characteristics of the plasmid, such as selectable marker and open reading frames, are shown.

### DEPOSIT OF MICROORGANISMS

The plasmids pM5A16T20 and pM7A16LLC34 were deposited on Apr. 20, 2017, under accession numbers DSM 32496 and DSM 32497, respectively, at the DSMZ—Deutsche Sammlung von Mikroorganismen and Zellkulturen, Inhoffenstraße 7 B, D-38124 Braunschweig, Federal Republic of Germany.

### SEQUENCE LISTING

The nucleic and amino acid sequences depicted in the accompanying sequence listing are shown using the standard letter abbreviations and codes applied conventionally in the art. Only one strand of each nucleic acid sequence is shown, but the complementary strand is understood as included by any reference to the displayed strand. In the accompanying sequence listing:

SEQ ID NO:1 is the amino acid sequence of the D1 domain of the human CD4 receptor.

SEQ ID NO:2 is the amino acid sequence of the D2 domain of the human CD4 receptor.

SEQ ID NO:3 is the amino acid sequence of the Fc portion of the human IgG1.

SEQ ID NO:4 is the amino acid sequence of the linker polypeptide GGGGS.

SEQ ID NO:5 is the amino acid sequence of the N-terminal region of the human CCR5 receptor.

SEQ ID NO:6 is the amino acid sequence of the M19 linker peptide.

SEQ ID NO:7 is the amino acid sequence of the M20 linker peptide.

SEQ ID NO:8 is the amino acid sequence of the M21 linker peptide.

SEQ ID NO:9 is the amino acid sequence of the M22 linker peptide.

SEQ ID NO:10 is the amino acid sequence of the T-20 polypeptide.

SEQ ID NO:11 is the amino acid sequence of the C34 polypeptide.

SEQ ID NO:12 is the amino acid sequence of the EHO polypeptide.

SEQ ID NO:13 is the amino acid sequence of the Fc portion of the human IgG1 bearing the AM set of mutations (i.e. G236A, S239D, A330L and I332E point mutations).

SEQ ID NO:14 is the amino acid sequence of the Fc portion of the human IgG1 bearing the A12 set of mutations (i.e. F243L, R292P and Y300L point mutations).

SEQ ID NO:15 is the amino acid sequence of the Fc portion of the human IgG1 bearing the A14 set of mutations (i.e. F243L, R292P and P396L point mutations).

SEQ ID NO:16 is the amino acid sequence of the Fc portion of the human IgG1 bearing the A16 set of mutations (i.e. F243L, R292P, Y300L and P396L point mutations).

SEQ ID NO:17 is the amino acid sequence of the Fc portion of the human IgG1 bearing the A18 set of mutations (i.e. F243L, R292P, Y300L, P396L and V305I point mutations).

SEQ ID NO:18 is the amino acid sequence of the Fc portion of the human IgG1 bearing the A41 set of mutations (i.e. S298A, E333A and K334A point mutations).

SEQ ID NO:19 is the amino acid sequence of the Fc portion of the human IgG1 bearing the LL set of mutations (i.e. M428L and N434S point mutations).

SEQ ID NO:20 is the amino acid sequence of the Fc portion of the human IgG1 bearing the AM+LL set of mutations (i.e. G236A, S239D, A330L, I332E, M428L and N434S point mutations).

SEQ ID NO:21 is the amino acid sequence of the Fc portion of the human IgG1 bearing the A12+LL set of mutations (i.e. F243L, R292P, Y300L, M428L and N434S point mutations).

SEQ ID NO:22 is the amino acid sequence of the Fc portion of the human IgG1 bearing the A14+LL set of mutations (i.e. F243L, R292P, P396L, M428L and N434S point mutations).

SEQ ID NO:23 is the amino acid sequence of the Fc portion of the human IgG1 bearing the A16+LL set of mutations (i.e. F243L, R292P, Y300L, P396L, M428L and N434S point mutations).

SEQ ID NO:24 is the amino acid sequence of the Fc portion of the human IgG1 bearing the A18+LL set of mutations (i.e. F243L, R292P, Y300L, P396L, V305I, M428L and N434S point mutations).

SEQ ID NO:25 is the amino acid sequence of the Fc portion of the human IgG1 bearing the A41+LL set of mutations (i.e. S298A, E333A, K334A, M428L and N434S point mutations).

SEQ ID NO:26 is the amino acid sequence of the Fc portion of the human IgG1 bearing the A16_1 and LL sets of mutations (i.e. S239D, F243L, R292P, Y300L, P396L, M428L and N434S point mutations).

SEQ ID NO:27 is the amino acid sequence of the Fc portion of the human IgG1 bearing the A16_2 and LL sets of mutations (i.e. F243L, R292P, Y300L, K322A, P396L, M428L and N434S point mutations).

SEQ ID NO:28 is the amino acid sequence of the Fc portion of the human IgG1 bearing the A16_3 and LL sets of mutations (i.e. F243L, R292P, Y300L, I332E, P396L, M428L and N434S point mutations).

SEQ ID NO:29 is the amino acid sequence of the Fc portion of the human IgG1 bearing the A16_4 and LL sets of mutations (i.e. F243L, R292P, Y300L, K322A, I332E, P396L, M428L and N434S point mutations).

SEQ ID NO:30 is the amino acid sequence of the Fc portion of the human IgG1 bearing the A16_5 and LL sets of mutations (i.e. F243L, R292P, Y300L, E333A, K334A, P396L, M428L and N434S point mutations).

SEQ ID NO:31 is the amino acid sequence of the Fc portion of the human IgG1 bearing the A16_6 and LL sets of mutations (i.e. S239D, F243L, R292P, Y300L, K322A, I332E, K334A, P396L, M428L and N434S point mutations).

SEQ ID NO:32 is the amino acid sequence of the Fc portion of the human IgG1 bearing the A41_1 and LL sets of mutations (i.e. S239D, F243L, R292P, S298A, E333A, K334A, M428L and N434S point mutations).

SEQ ID NO:33 is the amino acid sequence of the Fc portion of the human IgG1 bearing the A41, A16 and LL sets of mutations (i.e. F243L, R292P, S298A, E333A, K334A, M428L and N434S point mutations).

SEQ ID NO:34 is the nucleotide sequence of the D1 domain of the human CD4 receptor.

SEQ ID NO:35 is the nucleotide sequence of the D2 domain of the human CD4 receptor.

SEQ ID NO:36 is the nucleotide sequence of the Fc portion of the human IgG1.

SEQ ID NO:37 is the nucleotide sequence of the linker polypeptide.

SEQ ID NO:38 is the nucleotide sequence of the 5′ terminal region of the human CCR5 receptor.

SEQ ID NO:39 is the nucleotide sequence of the M19 linker peptide.

SEQ ID NO:40 is the nucleotide sequence of the M20 linker peptide.

SEQ ID NO:41 is the nucleotide sequence of the M21 linker peptide.

SEQ ID NO:42 is the nucleotide sequence of the M22 linker peptide.

SEQ ID NO:43 is the nucleotide sequence of the T-20 polypeptide.

SEQ ID NO:44 is the nucleotide sequence of the C34 polypeptide.

SEQ ID NO:45 is the nucleotide sequence of the EHO polypeptide.

SEQ ID NO:46 is the nucleotide sequence of the Fc portion of the human IgG1 bearing the AM set of mutations (i.e. G236A, S239D, A330L and I332E point mutations).

SEQ ID NO:47 is the nucleotide sequence of the Fc portion of the human IgG1 bearing the A12 set of mutations (i.e. F243L, R292P and Y300L point mutations).

SEQ ID NO:48 is the nucleotide sequence of the Fc portion of the human IgG1 bearing the A14 set of mutations (i.e. F243L, R292P and P396L point mutations).

SEQ ID NO:49 is the nucleotide sequence of the Fc portion of the human IgG1 bearing the A16 set of mutations (i.e. F243L, R292P, Y300L and P396L point mutations).

SEQ ID NO:50 is the nucleotide sequence of the Fc portion of the human IgG1 bearing the A18 set of mutations (i.e. F243L, R292P, Y300L, P396L and V305I point mutations).

SEQ ID NO:51 is the nucleotide sequence of the Fc portion of the human IgG1 bearing the A41 set of mutations (i.e. S298A, E333A and K334A point mutations).

SEQ ID NO:52 is the nucleotide sequence of the Fc portion of the human IgG1 bearing the LL set of mutations (i.e. M428L and N434S point mutations).

SEQ ID NO:53 is the nucleotide sequence of the Fc portion of the human IgG1 bearing the AM+LL set of mutations (i.e. G236A, S239D, A330L, I332E, M428L and N434S point mutations).

SEQ ID NO:54 is the nucleotide sequence of the Fc portion of the human IgG1 bearing the A12+LL set of mutations (i.e. F243L, R292P, Y300L, M428L and N434S point mutations).

SEQ ID NO:55 is the nucleotide sequence of the Fc portion of the human IgG1 bearing A14+LL the set of mutations (i.e. F243L, R292P, P396L, M428L and N434S point mutations).

SEQ ID NO:56 is the nucleotide sequence of the Fc portion of the human IgG1 bearing the A16+LL set of mutations (i.e F243L, R292P, Y300L, P396L, M428L and N434S point mutations).

SEQ ID NO:57 is the nucleotide sequence of the Fc portion of the human IgG1 bearing the A18+LL set of mutations (i.e F243L, R292P, Y300L, P396L, V305I, M428L and N434S point mutations).

SEQ ID NO:58 is the nucleotide sequence of the Fc portion of the human IgG1 bearing the A41+LL set of mutations (i.e. S298A, E333A, K334A, M428L and N434S point mutations).

SEQ ID NO:59 is the nucleotide sequence of the Fc portion of the human IgG1 bearing the A16_1 and LL sets of mutations (i.e. S239D, F243L, R292P, Y300L, P396L, M428L and N434S point mutations).

SEQ ID NO:60 is the nucleotide sequence of the Fc portion of the human IgG1 bearing the A16_2 and LL sets of mutations (i.e. F243L, R292P, Y300L, K322A, P396L, M428L and N434S point mutations).

SEQ ID NO:61 is the nucleotide sequence of the Fc portion of the human IgG1 bearing the A16_3 and LL sets of mutations (i.e. F243L, R292P, Y300L, I332E, P396L, M428L and N434S point mutations).

SEQ ID NO:62 is the nucleotide sequence of the Fc portion of the human IgG1 bearing the A16_4 and LL sets of mutations (i.e. F243L, R292P, Y300L, K322A, I332E, P396L, M428L and N434S point mutations).

SEQ ID NO:63 is the nucleotide sequence of the Fc portion of the human IgG1 bearing the A16_5 and LL sets of mutations (i.e. F243L, R292P, Y300L, E333A, K334A, P396L, M428L and N434S point mutations).

SEQ ID NO:64 is the nucleotide sequence of the Fc portion of the human IgG1 bearing the A16_6 and LL sets of mutations (i.e. S239D, F243L, R292P, Y300L, K322A, I332E, K334A, P396L, M428L and N434S point mutations).

SEQ ID NO:65 is the nucleotide sequence of the Fc portion of the human IgG1 bearing the A41_1 and LL sets of mutations (i.e. S239D, F243L, R292P, S298A, E333A, K334A, M428L and N434S point mutations).

SEQ ID NO:66 is the nucleotide sequence of the Fc portion of the human IgG1 bearing the A41, A16 and LL sets of mutations (i.e. F243L, R292P, S298A, E333A, K334A, M428L and N434S point mutations).

### DETAILED DESCRIPTION

The present invention relates to anti-HIV Fc-fusion protein derivatives with enhanced dual antiviral and immunomodulatory activities. The Fc-fusion protein derivatives of the present invention are characterized for having an increased ability to (i) block the entry of human immunodeficiency virus (HIV) into host cells and (ii) elicit effector functions through the activation of natural killer (NK) cells. The Fc-fusion protein derivatives of the present invention are also characterized for having (iii) a high production on mammalian cells and (iv) an extended activity in vivo.

### 1. Definitions of General Terms and Expressions

The term “AC10”, as used herein, refers to a HIV primary isolate characterized by its resistance to anti-CD4 binding site antibodies. AC10 is classified as a tier 2 isolate. See Seaman M, et al., J. Virol. 2010; 84:1439-1452.

The term “adeno-associated virus” or “AAV”, as used herein, refers to a virus member of the Parvoviridae family which comprises a linear, single-stranded DNA genome of about 5,000 nucleotides. At least 11 recognized serotypes of AAV (AAV1-11) are known in the art.

The term “AAV vector”, as used herein, refers to a nucleic acid having an AAV 5′ inverted terminal repeat (ITR) sequence and an AAV 3′ ITR flanking a polypeptide-coding sequence operably linked to transcription regulatory elements (e.g. promoters, enhancers) and a polyadenylation sequence. The AAV vector may include, optionally, one or more introns inserted between exons of the polypeptide-coding sequence. See Samulski J, et al., Annu. Rev. Virol. 2014; 1:427-451.

The term “AIDS”, as used herein, refers to the symptomatic phase of HIV infection, and includes both Acquired Immune Deficiency Syndrome (commonly known as AIDS) and “ARC,” or AIDS-Related Complex. See Adler M, et al., Brit. Med. J. 1987; 294: 1145-1147. The immunological and clinical manifestations of AIDS are well known in the art and include, for example, opportunistic infections and cancers resulting from immune deficiency.

The term “amino acid”, as used herein, refers to naturally occurring and synthetic amino acids, as well as amino acid analogs and amino acid mimetics that function in a manner similar to the naturally occurring amino acids. Amino acids may be referred to herein by either their commonly known three-letter symbols or by the one-letter symbols recommended by the IUPAC-IUB Biochemical Nomenclature Commission. Nucleotides, likewise, may be referred to by their commonly accepted single-letter codes.

The term “antibody drug conjugate” or “ADC”, as used herein, refers to an antibody, antigen-binding antibody fragment, Fc-fusion protein derivative, antibody complex or antibody fusion protein that is conjugated to a therapeutic agent. Conjugation may be covalent or non-covalent. Preferably, conjugation is covalent.

The term “antiretroviral therapy” or “AT”, as used herein, refers to the administration of one or more antiretroviral drugs (i.e. HIV antiretrovirals) to inhibit the replication of HIV. Typically, AT involves the administration of at least one antiretroviral agent (or, commonly, a cocktail of antiretrovirals) such as nucleoside reverse transcriptase inhibitor (e.g. zidovudine (AZT, lamivudine (3TC) and abacavir), non-nucleoside reverse transcriptase inhibitor (e.g. nevirapine and efavirenz) and protease inhibitor (e.g. indinavir, ritonavir and lopinavir). The term Highly Active Antiretroviral Therapy (“HAART”) refers to treatment regimens designed to suppress aggressively HIV replication and disease progression. HAART usually consists of three or more different drugs, such as, for example, two nucleoside reverse transcriptase inhibitors and a protease inhibitor.

The term “binding efficacy”, as used herein, refers to the affinity of a molecule to the CD4 receptor and, preferably, the D1 and D2 domains of said receptor. In the context of the invention, “affinity” means the strength with which a Fc-fusion protein derivative binds, for example, to the CD4 binding site of gp120. As used herein, the term “binding” or “specifically binding”, refers to the interaction between binding pairs (e.g. two proteins or compounds, preferably between (i) the CD4 binding site of gp120 and (ii) the CD4 receptor or the D1 and D2 domains of the CD4 receptor. In some embodiments, the interaction has an affinity constant of at most 10⁻⁶ moles/liter, at most 10⁻⁷ moles/liter, or at most 10⁻⁸ moles/liter. In general, the phrase “binding” or “specifically binding” refers to the specific binding of one compound to another, wherein the level of binding, as measured by any standard assay, is statistically significantly higher than the background control for the assay.

The term “C34”, as used herein, refers to a gp41-derived polypeptide of SEQ ID NO:11 covering part of the HR2 region of gp41. See Eggink D, et al., J. Virol. 2008; 82(13):6678-6688.

The term “CCR5”, as used herein, refers to the human C—C chemokine receptor 5, also known as CD195, a 7-transmembrane domain receptor coupled to G proteins. CCR5 binds to different chemokines and acts as the coreceptor of HIV Env during the process of HIV entry into target cells. The HIV coreceptor function involves different regions of CCR5; however, the first interaction is established between the N-terminal extracellular region of CCR5 and the coreceptor binding site of HIV Env located in the gp120 subunit. See Lagenaur L, et al., Retrovirology 2010; 7:11. The complete protein sequence for human CCR5 has the UniProt accession number P51681 (Aug. 18, 2015).

The term “CD4” or “CD4 receptor”, as used herein, refers to a cluster of differentiation 4, a glycoprotein expressed on the surface of T helper cells, monocytes, macrophages and dendritic cells. CD4 assists the T cell receptor (TCR) in its joining with an antigen-presenting cell. Using its portion that resides inside the T cell, CD4 amplifies the signal generated by the TCR by recruiting an enzyme, known as the tyrosine kinase lck, which is essential for activating many molecules involved in the signaling cascade of an activated T cell. The complete protein sequence for human CD4 has the UniProt accession number P01730 (Jun. 18, 2012).

The term “codon optimized”, as used herein, refers to the alteration of codons in nucleic acids to reflect the typical codon usage of the host organism to improve the expression of a reference polypeptide without altering its amino acid sequence. There are several methods and software tools known in the art for codon optimization. See Narum D, et al., Infect. Immun. 2001; 69(12):7250-7253), Outchkourov N, et al., Protein Expr. Purif. 2002; 24(1):18-24, Feng L, et al., Biochemistry 2000; 39(50):15399-15409 and Humphreys D, et al., Protein Expr. Purif. 2000; 20(2):252-264.

The term “complement”, as used herein, refers to a part of the innate immune system that enhances (complements) the ability of antibodies and phagocytic cells to clear microbes and damaged cells from an organism, promotes inflammation and attacks the pathogen's plasma membrane. See Janeway C, et al., “The complement system and innate immunity”, Immunobiology: The Immune System in Health and Disease (Garland Science, N.Y., US, 2001).

The term “comprising” or “comprises”, as used herein, discloses also “consisting of” according to the generally accepted patent practice.

The term “EHO”, as used herein, refers to the peptide C34EHO, a sequence of the HR2 fragment of the transmembrane subunit of the HIV-2 isolate EHO of SEQ ID NO:12.

The term “Env” or “gp160”, as used herein, refers to a glycoprotein having either the antigenic specificity or the biological function of the outer envelope protein (Env) of HIV and encompassing two subunits, the gp120 and the gp41 glycoproteins. Exemplary sequences of wild-type (wt) gp160 polypeptides are available. See GenBank accession nos. AAB05604 and AAD12142.

The term “fragment crystallizable region” or “Fc region”, as used herein, refers to the tail region of an antibody that interacts with cell surface receptors called Fc receptors and some proteins of the complement system.

The expression “functionally equivalent variant”, as used herein, refers to: (i) a polypeptide resulting from the modification, deletion or insertion or one or more amino acids and which substantially preserves the activity of its reference polypeptide and (ii) a polynucleotide resulting from the modification, deletion or insertion or one or more bases and which substantially preserves the activity of the polypeptide expressed by the reference nucleic acid. Functionally equivalent variants contemplated in the context of the present invention, include polypeptides which show at least 60%, 70%, 80%, 85%, 90%, 92%, 94%, 96%, 98%, 99% of similarity or identity with sequences SEQ ID Nos:1-33 or polynucleotides which show at least 60%, 70%, 80%, 85%, 90%, 92%, 94%, 96%, 98%, 99% of similarity or identity with sequences SEQ ID Nos:34-66. The degree of identity or similarity between two polypeptides or two polynucleotides is determined by using computer-implemented algorithms and methods that are widely known in the art. The identity and similarity between two sequences of amino acids is preferably determined using the BLASTP algorithm. See Altschul S, et al., “BLAST Manual” (NCBI NLM NIH, Bethesda, Md., USA, 2001).

The term “fusion protein”, as used herein, relates to proteins generated by gene technology which consist of two or more functional domains derived from different proteins. A fusion protein may be obtained by conventional means (e.g. by means of gene expression of the nucleotide sequence encoding for said fusion protein in a suitable cell).

The term “gp41”, as used herein, refers to human immunodeficiency virus-1 envelope glycoprotein gp41. Gp41 is a subunit which forms the Env glycoprotein of HIV-1 together with gp120. Env is a trimer composed of three external subunits (gp120) and three transmembrane subunits (gp41). The extracellular moiety of gp41 protein contains three essential functional regions: a fusion peptide (FP), a N-terminal heptad repeat (HR1) and a C-terminal heptad repeat (HR2). The HR1 and HR2 regions contain a number of leucine zipper-like motifs which have tendency to form coiled structures. See Peisajovich S, Shai Y, Biochem. Biophys. Acta 2003; 1614:122-129; Suarez T, et al., FEBS Lett. 2000; 477:145-149; Chan D, et al., Cell 1997; 89:263-273. As used herein, “gp41” refers also to the envelope transmembrane glycoprotein of other HIV types (e.g. HIV-2, SHIV, SIV) besides HIV-1. The nucleic acid and amino acid sequences of many HIV gp-41 are readily available to the public. See HIV Sequence Database, hiv.lanl.gov/content/sequence/HIV/mainpage, April 2017.

The term “gp41 inhibitors”, as used herein, include a series of polypeptides of different length that cover the HR2 region of gp41. These inhibitors include, but are not limited to, the T-20, C34, T-1249, T-2635 and EHO gp41-derived polypeptides.

The expression “gp41-derived polypeptide”, as used herein, refers to a polypeptide derived from the heptad repeat 1 (HR1) or the heptad repeat 2 (HR2) motifs of gp41. The gp41 HR1 and HR2 sequences are well known in the art. See Lupas A, Trends Biochem. Sci. 1996; 21:375-382 and Chambers P, et al., J. Gen. Virol. 1990; 71:3075-3080. Preferably, the gp41-derived polypeptide originates from HR2. The gp41-derived polypeptides may contain additional exogenous amino acid located at their N- or C-terminals. Preferably, the exogenous amino acids are less than 10, more preferably, less than 5, and, most preferably, less than 3.

The term “gp120”, as used herein, refers to a glycoprotein having either the antigenic specificity or the biological function of the outer envelope protein (env) of HIV. A “gp120 protein” is a molecule derived from a gp120 region of an Env polypeptide. The amino acid sequence of gp120 is approximately 511 amino acids. Gp120 is a heavily N-glycosylated protein with an apparent molecular weight of 120 kD. Gp120 contains five relatively conserved domains (C1-C5) interspersed with five variable domains (V1-V5). The variable domains contain extensive amino acid substitutions, insertions and deletions. A “gp120 polypeptide” includes both single subunits and multimers. The gp41 portion is anchored in (and spans) the membrane bilayer of the virion, while the gp120 segment protrudes into the surrounding environment. The receptor binding domain of gp120 is localized to N-terminal half of the protein. This is followed by a proline rich region (PRR), which behaves either as a hinge or trigger to communicate receptor binding to the fusion machinery. The C-terminus of gp120 is highly conserved and interacts with gp41. See GenBank accession nos. AAB05604 and AAD12142.

The term “HIV”, as used herein, include HIV-1 and HIV-2, SHIV and SIV. “HIV-1” means the human immunodeficiency virus type-1. HIV-1 includes, but is not limited to, extracellular virus particles and the forms of HIV-1 associated with HIV-1 infected cells. The HIV-1 virus may represent any of the known major subtypes (Classes A, B, C, D E, F, G and H) or outlying subtype (Group O) including laboratory strains and primary isolates. “HIV-2” means the human immunodeficiency virus type-2. HIV-2 includes, but is not limited to, extracellular virus particles and the forms of HIV-2 associated with HIV-2 infected cells. The term “SIV” refers to simian immunodeficiency virus which is an HIV-like virus that infects monkeys, chimpanzees, and other nonhuman primates. SIV includes, but is not limited to, extracellular virus particles and the forms of SIV associated with SIV infected cells.

The term “HIV exposure”, as used herein, refers to 16uclidet of an uninfected subject with a subject having an HIV infection or AIDS, or the contact with body fluids from such HIV-infected subject, in which such fluids from the infected subject contact a mucous membrane, a cut or abrasion in the tissue (e.g. needle stick, unprotected sexual intercourse), or other surface of the uninfected subject in such a way that the virus could be transmitted from the infected subject or infected subject's body fluids to the uninfected subject.

The term “HIV infection”, as used herein, refers to indications of the presence of the HIV virus in an individual including asymptomatic seropositivity, AIDS-related complex (ARC), and acquired immunodeficiency syndrome (AIDS).

The term “IC₅₀”, as used herein, refers to the amount of a particular active agent required for inhibiting 50% of a given biological process or component of a biological process (i.e. an enzyme, cell, cell receptor or microorganism).

The terms “identical” or percent “identity” in the context of two or more nucleic acids or polypeptides refer to two or more sequences or subsequences that are the same or have a specified percentage of nucleotides or amino acid residues that are the same when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art which can be used to obtain alignments of amino acid or nucleotide sequences. Examples of algorithms suitable for determining sequence similarity include, but are not limited to, the BLAST, Gapped BLAST, and BLAST 2.0, WU-BLAST-2, ALIGN, and ALIGN-2 algorithms. See Altschul S, et al., Nuc. Acids Res. 1977; 25:3389-3402, Altschul S, et al., J. Mol. Biol. 1990; 215:403-410, Altschul S, et al., Meth. Enzymol. 1996; 266:460-480, Karlin S, et al., Proc. Natl. Acad. Sci. USA1990; 87:2264-2268, Karlin S, et al., Proc. Natl. Acad. Sci. USA1993; 90:5873-5877, Genentech Corp, South San Francisco, Calif., US, blast.ncbi.nlm.nih.gov/Blast.cgi, April 2017. Methods of alignment of sequences for comparison are well known in the art. Optimal alignment of sequences for comparison can be conducted, for instance, by the Smith-Waterman local homology algorithm, by the Needleman-Wunsch homology alignment algorithm, by the Pearson-Lipman similarity search method, by computerized implementations of these algorithms or by manual alignment and visual inspection. See Smith T, et al., Adv. Appl. Math. 1981; 2:482-489, Needleman S, et al., J. Mol. Biol. 1970; 48:443-453, Pearson W, et al., Lipman D, Proc. Natl. Acad. Sci. USA1988; 85:2444-2448, the GAP, BESTFIT, FASTA and TFASTA programs, Wisconsin Genetics Software Package, Genetics Computer Group, Madison, Wis., USA; Ausubel F, et al., Eds., “Short Protocols in Molecular Biology”, 5^{th} Ed. (John Wiley and Sons, Inc., New York, N.Y., USA, 2002).

The term “kit”, as used herein, refers to a product containing the different reagents necessary for carrying out the uses and methods of the invention which is packed so as to allow their transport and storage. Materials suitable for packing the components of the kit include crystal, plastic (e.g. polyethylene, polypropylene, polycarbonate), bottles, vials, paper or envelopes.

The term “neutralizing antibody”, as used herein, is any antibody, antigen-binding fragment or Fc-fusion protein derivative that binds to an extracellular molecule (e.g. a protein or a protein domain in the surface of a pathogenic virus) and interferes with the ability of the extracellular molecule to infect a cell or modulate its activity. Typically, the Fc-fusion protein derivatives of the invention can bind to the surface of the extracellular molecule and are able to inhibit its coupling to a cell by at least 99%, 95%, 90%, 85%, 80%, 75%, 70%, 60%, 50%, 45%, 40%, 35%, 30%, 25%, 20% or 10% relative to the attachment of the extracellular molecule to the cell in the absence of said Fc-fusion protein derivatives or in the presence of a negative control. Methods for confirming whether a Fc-fusion protein derivative is neutralizing have been described in the art. See Li M, et al., J. Virol. 2005; 79:10108-10125, Wei X, et al., Nature 2003; 422:307-312, and Montefiori D, Curr. Protoc. Immunol. 2005; January, Chapter 12:Unit 12.11. In the context of the invention, the pathogen is preferably HIV, and more specifically, the gp120 protein of the HIV viral envelope. In particular, the term “HIV neutralizing antibody” refers to a Fc-fusion protein derivative with affinity to the CD4 binding site of gp120 such as IgGb12. The term “neutralizing antibodies” includes the subclass of bnAbs. As used herein, “broadly neutralizing antibody” or “bnAb” is understood as an antibody obtained by any method that, when delivered at an effective dose, can be used as a therapeutic agent for the prevention or treatment of HIV infection or AIDS against more than 7 strains of HIV, preferably more than 8, 9, 10, 11, 12, 13, 14, 15, 16, 17, 18, 19, 20 or more strains of HIV.

The term “NK cell”, as used herein, refers to a “Natural Killer cell”, a type of cytotoxic lymphocyte critical to the innate immune system. NK cells provide rapid responses to virally infected cells and respond to tumor formation, acting at around 3 days after infection. Typically, immune cells detect HLA presented on infected cell surfaces, triggering cytokine release causing lysis or apoptosis. NK cells are unique, however, as they have the ability to recognize stressed cells in the absence of antibodies and HLA, allowing for a much faster immune reaction. NK cells are defined as large granular lymphocytes and constitute the third kind of cells differentiated from the common lymphoid progenitor generating B and T lymphocytes. NK cells are known to differentiate and mature in the bone marrow, lymph node, spleen, tonsils and thymus where they enter into circulation. NK cells express usually the surface markers CD16 (FcγRIII) and CD56 in humans.

The terms “NL4-3” and “BaL”, as used herein, refer to two different HIV isolates commonly used in the laboratory. The NL4-3 isolate was cloned from NY5 and LAV proviruses. See Adachi A, et al., J. Virol. 1986; 59:284-291. The BaL isolate was obtained from a primary culture of adherent cells grown from explanted lung tissue. See Gartner S, et al., Science 1986; 233:215-219.

The terms “nucleic acid”, “polynucleotide” and “nucleotide sequence”, as used interchangeably herein, relate to any polymeric form of nucleotides of any length and composed of ribonucleotides or deoxyribonucleotides. The terms include both single-stranded and double-stranded polynucleotides, as well as modified polynucleotides (e.g. methylated, protected). Typically, the nucleic acid is a “coding sequence” which, as used herein, refers to a DNA sequence that is transcribed and translated into a polypeptide in a host cell when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, prokaryotic sequences, cDNA from eukaryotic mRNA, genomic DNA sequences from eukaryotic (e.g. mammalian) DNA, and even synthetic DNA sequences. A transcription termination sequence will usually be located 3′ to the coding sequence.

The term “operably linked”, as used herein, means that the nucleotide sequence of interest is linked to the regulatory sequence(s) in a manner that allows for expression of the nucleotide sequence (e.g. in an in vitro transcription/translation system or in a host cell when the vector is introduced into the host cell). See Auer H, Nature Biotechnol. 2006; 24: 41-43.

The expression “panel of HIV isolates”, as used herein, refers to a collection of reference HIV isolates designed for use as Env-pseudotyped viruses to facilitate standardized tier ⅔ assessments of neutralizing antibody responses. See Mascola R, et al., J. Virol. 2005; 79(16):10103. The pseudoviruses exhibit a neutralization phenotype that is typical of most primary HIV-1 isolates. The gp160 genes were cloned from sexually acquired, acute/early infections and comprise a wide spectrum of genetic, antigenic and geographic diversity within subtype B. These clones use CCR5 as co-receptor. See Li, et al., J. Virol. 2005; 79(16):10108-10125.

The expression “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and, intrasternal injection and infusion.

The expression “pharmaceutically acceptable carrier”, as used herein, includes any and all solvents, dispersion media, coatings, antibacterial and antifungal agents, isotonic and absorption delaying agents that are physiologically compatible with the Fc-fusion protein derivatives, nucleic acids, vectors and host cells of the invention.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The terms “prevent,” “preventing” and “prevention”, as used herein, refer to inhibiting the inception or decreasing the occurrence of a disease in a subject. The prevention may be complete (e.g. the total absence of pathological cells in a subject). The prevention may also be partial, such as, for example, lowering the occurrence of pathological cells in a subject. Prevention also refers to a reduced susceptibility to a clinical condition. Within the context of the present invention, the terms “prevent,” “preventing” and “prevention”, refer specifically to averting or reducing the probability of HIV infection in a subject sustaining HIV exposure.

The term “sample”, as used herein, refers to any biofluid and, in particular, blood, serum, plasma, lymph, saliva, peripheral blood cells or tissue cells serum, semen, sputum, cephalorachidian liquid (CRL), tears, mucus, sweat, milk or brain extracts obtained from a subject. The bodily tissue may comprise thymus, lymph node, spleen, bone marrow or tonsil tissue. The term “sample” refers also to non-biological samples (e.g. obtained from water, beverages).

The term “subject”, as used herein, refers to an individual, plant or animal, such as a human, a nonhuman primate (e.g. chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. The term “subject” encompasses an embryo and a fetus. In a preferred embodiment, the subject is a human.

The term “T-1249”, a used herein, refers to a gp41-derived polypeptide covering part of the HR2 region of gp41. See Eggink, 2008, supra.

The term “T-20”, a used herein, refers to a gp41-derived polypeptide of SEQ ID NO:10 covering part of the HR2 region of gp41, also known as enfuvirtide. See CAS [159519-65-0] and U.S. Pat. No. 5,464,933. This polypeptide has antiviral activity in the nanomolar range and has been used in therapy against HIV infection. See Zhang D, et al., Expert Opin Ther Pat. 2015; 25:159-173 and Eggink, 2008, supra.

The term “T-2635”, a used herein, refers to a gp41-derived polypeptide covering part of the HR2 region of gp41. See Eggink, 2008, supra.

The term “therapeutic agent” as used herein, refers to an atom, molecule or compound useful in the treatment or prevention of a disease. Examples of therapeutic agents include, but are not limited to, antibodies, antibody fragments, HIV antiretrovirals, drugs, cytotoxic agents, pro-apopoptotic agents, toxins, nucleases (e.g. DNAses and RNAses), hormones, immunomodulators, chelators, boron compounds, photoactive agents or dyes, radionuclides, oligonucleotides, interference RNA, siRNA, RNAi, anti-angiogenic agents, chemotherapeutic agents, cytokines, chemokines, prodrugs, enzymes, binding proteins, peptides or combinations thereof.

The term “therapeutically effective amount”, as used herein, refers to the dose or amount of the Fc-fusion protein derivatives, nucleic acids, vectors, pharmaceutical compositions of the invention or mixtures thereof that produces a therapeutic response or desired effect in a subject.

The terms “therapy” or “therapeutic”, a used herein, refer to the use of the Fc-fusion protein derivatives, nucleic acids, vectors, pharmaceutical compositions of the invention or mixtures thereof for either the treatment or prevention of a disease including, but not limited to, HIV and AIDS.

The term “treat” or “treatment”, as used herein, refers to the administration of a Fc-fusion protein derivative, nucleic acid, vector, host cell or pharmaceutical composition of the invention for controlling the progression of a disease after its clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival compared with the expected survival if treatment was not applied. Within the context of the present invention, the terms “treat” and “treatment” refer specifically to stopping or slowing the infection and destruction of healthy CD4+ T cells in a HIV infected subject. It also refers to the stopping and slowing of the onset of symptoms of the acquired immunodeficiency disease such as extreme low CD4+ T cell count and repeated infections by opportunistic pathogens. Beneficial or desired clinical results include, but are not limited to, an increase in absolute naïve CD4+ T cell count (range 10-3520), an increase in the percentage of CD4+ T cell over total circulating immune cells (range 1-50%), or an increase in CD4+ T cell count as a percentage of normal CD4+ T cell count in an uninfected subject (range 1-161%). “Treatment” can also mean prolonging survival of the infected subject as compared to expected survival if the subject does not receive any HIV targeted treatment.

The term “vector”, as used herein, refers to a nucleic acid molecule, linear or circular, that comprises a nucleic acid of the invention operably linked to additional segments that provide for its autonomous replication in a host cell or according to the expression cassette of the nucleic acid molecule.

### 2. Fc Fusion Protein Derivatives

The present invention refers to Fc-fusion protein derivatives which comprise from the N- to C-terminus:
(a) the D1 and D2 extracellular domains of a human CD4 receptor,
(b) the Fc portion of a human IgG1 comprising at least one of a G236A, S239D, A330L, I332E, F243L, R292P, S298A, Y300L, V305I, K322A, E333A, K334A, P396L, M428L or N434S point mutations,
(c) a moiety selected from the group consisting of (i) a linker polypeptide of sequence (GGGGS)ₙ wherein 1≤n≤10 (GGGGS=SEQ ID NO: 4), (ii) SEQ ID NO:5-9 and (iii) combinations thereof and
(d) a gp41-derived polypeptide.

The Fc-fusion protein derivatives of the invention are characterized for having increased antiviral and ADCC activities. The expression and duration of action (i.e. t_{1/2}) of the Fc-fusion protein derivatives of the invention are also higher than other comparable antibodies.

(a) The D1 and D2 Extracellular Domains of a Human CD4 Receptor

In one embodiment, the D1 domain of the Fc-fusion protein derivatives of the invention comprises amino acids 26-125 of the human CD4 receptor (i.e. UniProtKB database accession number P01730) or a functionally equivalent variant thereof. In another embodiment, the D2 domain of the Fc-fusion protein derivatives comprises amino acids 126-203 of the human CD4 receptor or a functionally equivalent variant thereof. Preferably, the D1 and D2 domains comprise sequences SEQ ID NO:1 and SEQ ID NO:2, respectively, or a functionally equivalent variant thereof.

(b) The Fc Portion of a Human IgG1

In an additional embodiment, the Fc portion of the human IgG1 comprise sets and subsets of combinations of mutations that comprises at least one of a F243L, R292P, S298A, Y300L, V305I, K322A, E333A, K334A, P396L, M428L or N434S point mutations or a functionally equivalent variant thereof. In a further embodiment, the Fc portion of the human IgG1 comprises (i) at least one of a G236A, S239D, A330L or I332E point mutations or a functionally equivalent variant thereof.
(i) M428L and N434S point mutations (LL set of mutations)

In an additional version of this embodiment, the Fc portion of the human IgG1 comprise at least one of a M428L or N434S point mutations or a functionally equivalent variant thereof. Preferably, the Fc portion of the human IgG1 comprises both M428L and N434S point mutations or a functionally equivalent variant thereof. The Fc portion of the human IgG1 comprising SEQ ID NO:19 or a functionally equivalent variant thereof is preferred.

In a further feature of this version, the Fc portion of the human IgG1 comprises (i) at least one of a G236A, S239D, A330L or I332E point mutations and (ii) at least one of a M428L or N434S point mutations or a functionally equivalent variant thereof. In one feature of this version, the Fc portion of the human IgG1 comprises (i) at least one of a G236A, S239D, A330L or I332E point mutations and a M428L point mutation or a functionally equivalent variant thereof. In another feature of this version, the Fc portion of the human IgG1 comprises (i) at least one of a G236A, S239D, A330L or I332E point mutations and a N434S point mutation or a functionally equivalent variant thereof. Preferably, the Fc portion of the human IgG1 comprises G236A, S239D, A330L and I332E point mutations or a functionally equivalent variant thereof. The Fc portion of the human IgG1 comprising SEQ ID NO:19 or a functionally equivalent variant thereof is preferred.

In another feature of this version, the Fc portion of the human IgG1 comprises at least one or more of a F243L or R292P point mutations or a functionally equivalent variant thereof. Preferably, the Fc portion of the human IgG1 comprises both F243L and R292P point mutations or a functionally equivalent variant thereof. In a supplementary feature, the Fc portion of the human IgG1 comprises at least one of a Y300L, P396L, S298A, E333A or K334A point mutations or a functionally equivalent variant thereof. The Fc portion of the human IgG1 comprising SEQ ID NO:21-25 or a functionally equivalent variant thereof are preferred.

In a further feature of this version, the Fc portion of the human IgG1 comprises a K322A point mutation. The Fc portion of the human IgG1 comprising SEQ ID NO:27, SEQ ID NO:29 or SEQ ID NO:31 or a functionally equivalent variant thereof are preferred
(ii) F243L, R292P, Y300L, P396L, S298A, E333A and K334A (A12, A14, A16 and A41 set of mutations)

In another version of this embodiment, the Fc portion of the human IgG1 comprises at least one or more of a F243L or R292P point mutations or a functionally equivalent variant thereof. Preferably, the Fc portion of the human IgG1 comprises both F243L and R292P point mutations or a functionally equivalent variant thereof. In a supplementary feature of this version, the Fc portion of the human IgG1 comprises at least one of a Y300L, P396L, S298A, E333A or K334A point mutations or a functionally equivalent variant thereof. The Fc portion of the human IgG1 comprising SEQ ID NO:14-18 or a functionally equivalent variant thereof are preferred.

In a further feature of this version, the Fc portion of the human IgG1 comprises (i) at least one of a G236A, S239D, A330L or I332E point mutations or a functionally equivalent variant thereof. Preferably, the Fc portion of the human IgG1 comprises G236A, S239D, A330L and I332E point mutations or a functionally equivalent variant thereof. In a further feature of this version, the Fc portion of the human IgG1 comprises a K322A point mutation.
(iii) K322A point mutation

In another version of this embodiment, the Fc portion of the human IgG1 a K322A point mutation or a functionally equivalent variant thereof.

In another feature of this version, the Fc portion of the human IgG1 comprises (i) at least one of a G236A, S239D, A330L or I332E point mutations and the K322 point mutation or a functionally equivalent variant thereof. Preferably, the Fc portion of the human IgG1 comprises G236A, S239D, A330L and I332E point mutations or a functionally equivalent variant thereof.

In a supplementary version of this embodiment, the Fc portion of the human IgG1 comprises at least one or more of a F243L or R292P point mutations or a functionally equivalent variant thereof. Preferably, the Fc portion of the human IgG1 comprises both F243L and R292P point mutations or a functionally equivalent variant thereof. In a further feature of this version, the Fc portion of the human IgG1 comprises at least one of a Y300L, P396L, S298A, E333A or K334A point mutations or a functionally equivalent variant thereof.

(c) Moiety

In another embodiment, the moiety of the Fc-fusion protein derivatives of the invention is selected from the group consisting of (i) a linker polypeptide of sequence (GGGGS)ₙ (SEQ ID NO:4) wherein 1≤n≤10, (ii) SEQ ID NO:5-9, (iii) a combination of (i) and (ii) and a functionally equivalent variant of (i), (ii) and (iii). In a further version of this embodiment, the moiety of the Fc-fusion protein derivatives of the invention is selected from the group consisting of (i) a linker polypeptide of sequence (GGGGS)ₙ (SEQ ID NO:4) wherein 1≤n≤10, (ii) SEQ ID NO:5, (iii) a combination of (i) and (ii) and a functionally equivalent variant of (i), (ii) and (iii). In an additional version of this embodiment, the moiety of the Fc-fusion protein derivatives of the invention is selected from the group consisting of (i) a linker polypeptide of sequence (GGGGS)ₙ (SEQ ID NO:4) wherein 1≤n≤10, (ii) SEQ ID NO:6, (iii) a combination of (i) and (ii) and a functionally equivalent variant of (i), (ii) and (iii). In another version of this embodiment, the moiety of the Fc-fusion protein derivatives of the invention is selected from the group consisting of (i) a linker polypeptide of sequence (GGGGS)ₙ (SEQ ID NO:4) wherein 1≤n≤10, (ii) SEQ ID NO:7, (iii) a combination of (i) and (ii) and a functionally equivalent variant of (i), (ii) and (iii). In a further version of this embodiment, the moiety of the Fc-fusion protein derivatives of the invention is selected from the group consisting of (i) a linker polypeptide of sequence (GGGGS)ₙ (SEQ ID NO:4) wherein 1≤n≤10, (ii) SEQ ID NO:8, (iii) a combination of (i) and (ii) and a functionally equivalent variant of (i), (ii) and (iii). In another version of this embodiment, the moiety of the Fc-fusion protein derivatives of the invention is selected from the group consisting of (i) a linker polypeptide of sequence (GGGGS)ₙ (SEQ ID NO:4) wherein 1≤n≤10, (ii) SEQ ID NO:9, (iii) a combination of (i) and (ii) and a functionally equivalent variant of (i), (ii) and (iii). In one version of this embodiment, the moiety comprises only the linker or the human CCR5 receptor sequence or a functionally equivalent variant thereof. In another version of this embodiment, the moiety comprises a combination of the linker and the human CCR5 receptor sequence or a functionally equivalent variant thereof. Preferably, the linker is attached to the C-terminus of the human CCR5 receptor sequence when a combination is employed. Preferably, the moiety comprises the linker only or a combination of the linker and the human CCR5 receptor sequence or a functionally equivalent variant thereof. Preferably, the human CCR5 receptor sequence comprises SEQ ID NO:5 or a functionally equivalent variant thereof. In one version of this embodiment, SEQ ID NO:5-9 or their functionally equivalent variants have been further modified to include alanine residues. Preferably, the linker modified with alanine residues is SEQ ID NO:8 or a functionally equivalent variant thereof.

(d) gp41 Polypeptide

In a further embodiment, the gp41-derived polypeptide comprises the T-20, T-4912, C34, T-2635 and EHO polypeptide, their combinations or a functionally equivalent variant thereof. Preferably, the gp41-derived polypeptide comprises the T-20, C34 and EHO polypeptides or a functionally equivalent variant thereof. More preferably, the T-20, C34 and EHO polypeptide comprise sequences SEQ ID NO:10, SEQ ID NO:11 and SEQ ID NO:12, respectively.

In an additional embodiment, the Fc-fusion protein derivatives of the invention comprise sequences SEQ ID NO:13-33 or a functionally equivalent variant thereof. Preferably, the Fc-fusion protein derivatives comprise sequences SEQ ID NO:19-33 or a functionally equivalent variant thereof. More preferably, the Fc-fusion protein derivatives comprises sequence SEQ ID NO:20 or a functionally equivalent variant thereof.

Preferably, the Fc-fusion protein derivatives of the invention comprise:
(1) (a) the D1 and D2 extracellular domains of a human CD4 receptor, (b) the Fc portion of a human IgG1 comprising G236A, S239D, A330L, I332E and M428L point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5 and (iii) combinations thereof, and (d) a T-20 polypeptide. The Fc portion of the human IgG1 may comprise further at least one of a F243L, R292P, Y300L, P396L, S298A, E333A or K334A point mutations.
(2) (a) the D1 and D2 extracellular domains of a human CD4 receptor, (b) the Fc portion of a human IgG1 comprising G236A, S239D, A330L, I332E and M428L point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5 and (iii) combinations thereof, and (d) a C34 polypeptide. The Fc portion of the human IgG1 may comprise further at least one of a F243L, R292P, Y300L, P396L, S298A, E333A or K334A point mutations.
(3) (a) the D1 and D2 extracellular domains of a human CD4 receptor, (b) the Fc portion of a human IgG1 comprising G236A, S239D, A330L, I332E and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5 and (iii) combinations thereof, and (d) a T-20 polypeptide. The Fc portion of the human IgG1 may comprise further at least one of a F243L, R292P, Y300L, P396L, S298A, E333A or K334A point mutations.
(4) (a) the D1 and D2 extracellular domains of a human CD4 receptor, (b) the Fc portion of a human IgG1 comprising G236A, S239D, A330L, I332E and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5 and (iii) combinations thereof, and (d) a C34 polypeptide. The Fc portion of the human IgG1 may comprise further at least one of a F243L, R292P, Y300L, P396L, S298A, E333A or K334A point mutations.
(5) (a) the D1 and D2 extracellular domains of a human CD4 receptor, (b) the Fc portion of a human IgG1 comprising G236A, S239D, A330L, I332E, M428L and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5 and (iii) combinations thereof, and (d) a T-20 polypeptide. The Fc portion of the human IgG1 may comprise further at least one of a F243L, R292P, Y300L, P396L, S298A, E333A or K334A point mutations.
(6) (a) the D1 and D2 extracellular domains of a human CD4 receptor, (b) the Fc portion of a human IgG1 comprising G236A, S239D, A330L, I332E, M428L and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5 and (iii) combinations thereof, and (d) a C34 polypeptide. The Fc portion of the human IgG1 may comprise further at least one of a F243L, R292P, Y300L, P396L, S298A, E333A or K334A point mutations.
(7) (a) the D1 and D2 extracellular domains of a human CD4 receptor, (b) the Fc portion of a human IgG1 comprising F243L, R292P, Y300L, K322A, I332E, P396L, M428L and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5-9 and (iii) combinations thereof, and (d) a C34 polypeptide. The Fc portion of the human IgG1 may comprise further at least one of a S298A, E333A or K334A point mutations. In another version of this embodiment, the moiety further includes alanine residues as in SEQ ID NO:8.
(8) (a) the D1 and D2 extracellular domains of a human CD4 receptor, (b) the Fc portion of a human IgG1 comprising F243L, R292P, Y300L, K322A, I332E, P396L, M428L and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5-9 and (iii) combinations thereof, and (d) a EHO polypeptide. The Fc portion of the human IgG1 may comprise further at least one of a S298A, E333A or K334A point mutations. In another version of this embodiment, the moiety includes further alanine residues as in SEQ ID NO:8.
(9) (a) the D1 and D2 extracellular domains of a human CD4 receptor, (b) the Fc portion of a human IgG1 comprising G236A, S239D, A330L, I332E and N434S point mutations, (c) a moiety selected from the group consisting of (i) (i) a linker polypeptide of sequence (GGGGS)ₙ wherein 1≤n≤10 (GGGGS=SEQ ID NO: 4), (ii) SEQ ID NO:5-9 and (iii) combinations thereof, and (d) a T-20 polypeptide. The Fc portion of the human IgG1 may comprise further at least one of a F243L, R292P, Y300L, P396L, S298A, E333A or K334A point mutations.
(10) (a) the D1 and D2 extracellular domains of a human CD4 receptor, (b) the Fc portion of a human IgG1 comprising G236A, S239D, A330L, I332E and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5-9 and (iii) combinations thereof, and (d) a C34 polypeptide. The Fc portion of the human IgG1 may comprise further at least one of a F243L, R292P, Y300L, P396L, S298A, E333A or K334A point mutations.
(11) (a) the D1 and D2 extracellular domains of a human CD4 receptor, (b) the Fc portion of a human IgG1 comprising G236A, S239D, A330L, I332E, M428L and N434S point mutations, (c) a moiety selected from the group consisting (i) a linker polypeptide of sequence (GGGGS)ₙ wherein 1≤n≤10 (GGGGS=SEQ ID NO: 4), (ii) SEQ ID NO:5-9 and (iii) combinations thereof, and (d) a T-20 polypeptide. The Fc portion of the human IgG1 may comprise further at least one of a F243L, R292P, Y300L, P396L, S298A, E333A or K334A point mutations.
(12) (a) the D1 and D2 extracellular domains of a human CD4 receptor, (b) the Fc portion of a human IgG1 comprising G236A, S239D, A330L, 1332E, M428L and N434S point mutations, (c) a moiety selected from the group consisting of (i) a linker polypeptide of sequence (GGGGS)ₙ wherein 1≤n≤10 (GGGGS=SEQ ID NO: 4), (ii) SEQ ID NO:5-9 and (iii) combinations thereof, and (d) a C34 polypeptide. The Fc portion of the human IgG1 may comprise further at least one of a F243L, R292P, Y300L, P396L, S298A, E333A or K334A point mutations.

The Fc-fusion protein derivatives of the invention are useful for preventing (i.e. neutralizing) the attachment of molecules (e.g. HIV) to the human CD4 receptor in cells expressing said cluster in their surface (e.g. T-helper cells, monocytes, macrophages, dendritic cells). Preferably, the Fc-fusion protein derivatives of the invention are utilized for preventing the attachment of gp160 proteins located in the viral envelope of HIV to human CD4 receptors found in T-helper cells. The neutralizing capacity of the Fc-fusion protein derivatives of the invention may be characterized by an IC₅₀ of 10 ng/mL or lower, and preferably, by an IC₅₀ of less than 5 ng/mL, less than 2.5 ng/mL, less than 1.25 ng/mL, less than 0.625 ng/mL, less than 0.312 μg/mL, less than 0.156 ng/mL, less than 0.07 ng/mL or less than 0.035 ng/mL.

In an additional embodiment, the Fc-fusion protein derivatives of the invention can be chemically modified by covalent conjugation to a polymer, for example, to increase their circulating half-life. Methods of attaching polypeptides to polymers are known in the art. See U.S. Pat. Nos. 4,766,106, 4,179,337, 4,495,285 and 4,609,546. Preferably, the polymers are polyoxyethylated polyols and polyethylene glycol (PEG). PEG is a water soluble polymer that has the general formula: R(O—CH₂—CH₂)ₙO—R where R can be hydrogen or a protective group such as an alkyl or alkanol group. Preferably, the protective group has between 1 and 8 carbons, more preferably it is methyl. Preferably, n is an integer between 1 and 1,000 and, more preferably between 2 and 500. PEG has a preferred average molecular weight between 1,000 and 40,000, more preferably between 2,000 and 20,000 and most preferably between 3,000 and 12,000.

In a further embodiment, the Fc-fusion protein derivatives of the invention are attached to a therapeutic agent to form an antibody drug conjugate (ADC). For instance, therapeutic agents used for the treatment of opportunistic diseases and conditions arising from or favored by the inception of AIDS such as, for example, Kaposi's sarcoma, may be treated with an ADC formed by a Fc-fusion protein derivative of the invention and interferon-α, a liposomal anthracycline (e.g. doxil) or paclitaxel. Further ADCs effective for the treatment of other opportunistic diseases, such as viral and bacterial infections (e.g. shingles, pneumonia, tuberculosis), skin diseases and other types of cancer (e.g. lymphoma) associated to AIDS, may also be devised by combining a Fc-fusion protein derivative of the invention and an appropriate therapeutic agent.

### 3. Nucleic Acids, Vectors and Host Cells

In another aspect, the present invention relates to nucleic acids encoding for the Fc-fusion protein derivative of the invention, and to the expression cassettes and vectors comprising said nucleic acids.

Preferably, the nucleic acids are polynucleotides, including, but not limited to, deoxyribonucleotides (DNA) and ribonucleotides (RNA) linked by internucleotide phosphodiester bond linkages. In a preferred embodiment, the nucleic acids of the invention comprise the polynucleotides encoding for the D1 and D2 extracellular domains of the human CD4 receptor (SEQ ID NO:34, SEQ ID NO:35), the Fc portion of the human IgG1 comprising G236A, S239D, A330L, 1332E, M428L and N434S point mutations (SEQ ID NO:53), the linker polypeptide (SEQ ID NO:37), the human CCR5 receptor (SEQ ID NO:38), and the T-20 polypeptide (SEQ ID NO:43), the C34 polypeptide (SEQ ID NO:44) or the EHO polypeptide (SEQ ID NO:45) or their functionally equivalent variants. Preferably, the nucleic acids of the invention encode for Fc-fusion protein derivatives comprising sequences SEQ ID NO:13-25 or a functionally equivalent variant thereof.

The functionally equivalent variants of the nucleic acids of the invention may be obtained by means of the insertion, deletion or substitution of one or several nucleotides with respect to their reference sequences. Preferably, the polynucleotides encoding for functionally equivalent variants of the nucleic acids of the invention are polynucleotides whose sequences allows them to hybridize in highly restrictive conditions with their nucleic acids of reference. Typical conditions of highly restrictive hybridization include incubation in 6×SSC (1×SSC: 0.15 M NaCl, 0.015 M sodium citrate) and 40% formamide at 42° C. during 14 hours, followed by one or several washing cycles using 0.5×SSC, 0.1% SDS at 60° C. Alternatively, highly restrictive conditions include those comprising a hybridization at a temperature of approximately 50-55° C. in 6×SSC and a final washing at a temperature of 68° C. in 1-3×SSC. Moderate restrictive conditions comprise hybridization at a temperature of approximately 50° C. until around 65° C. in 0.2 or 0.3 M NaCl, followed by washing at approximately 50° C. until around 55° C. in 0.2×SSC, 0.1% SDS (sodium dodecyl sulphate). In one further embodiment, the nucleic acids of the invention are codon optimized.

In another embodiment, a variant of a nucleic acid having at least 80%, 85%, 90%, 95%, or 99% similarity to its reference nucleic acid is used instead, wherein said variant encodes a Fc-fusion protein derivative of the invention or a functionally equivalent variant thereof.

The nucleic acids of the invention may require treatment with restriction enzymes for their ligation into a suitable vector (e.g. 1, 2 or 3 terminal nucleotides may be removed). In an additional embodiment, the invention relates to said nucleic acids, wherein they have been cut at each end with a restriction enzyme.

In another embodiment, the present invention relates to an expression cassette comprising a nucleic acid of the invention, a promoter sequence and a 3′-UTR and, optionally, a selection marker.

In yet another embodiment, the present invention relates to a vector comprising a nucleic acid of the invention. In an additional aspect of this embodiment, the nucleic acid of the invention is contained in an expression cassette comprised by said vector. Suitable vectors according to the present invention include, but are not limited to, prokaryotic vectors, such as pUC18, pUC19, and Bluescript plasmids and derivatives thereof, like the mp18, mp19, pBR322, pMB9, ColE1, pCR1 and RP4 plasmids; phages and shuttle vectors, such as pSA3 and pAT28 vectors; expression vectors in yeasts, such as 2-micron plasmid type vectors; integration plasmids; YEP vectors; centromeric plasmids and analogues; expression vectors in insect cells, such as the vectors of the pAC series and of the pVL series; expression vectors in plants, such as vectors of the pIBI, pEarleyGate, pAVA, pCAMBIA, pGSA, pGWB, pMDC, pMY, pORE series and analogues; and expression vectors in superior eukaryotic cells either based on viral vectors (e.g. adenoviruses, adeno-associated viruses, retroviruses, lentiviruses) as well as non-viral vectors, such as the pSilencer 4.1-CMV (Ambion®, Life Technologies Corp., Carlsbad, Calif., US), pcDNA3, pcDNA3.1, pcDNA3.1/hyg pHCMV/Zeo, pCR3.1, pEFl/His, pIND/GS, pRc/HCMV2, pSV40/Zeo2, pTRACER-HCMV, pUB6/V5-His, pVAXl, pZeoSV2, pCI, pSVL and pKSV-10, pBPV-1, pML2d, and pTDTl vectors. Preferably, the vector is a pcDNA3.1 vector. More preferably, the vector is pABT-5, pABT-7 and pABT-8.

In an additional embodiment, the viral vector is an AAV vector. AAV vectors encoding the Fc-fusion protein derivatives of the invention may be constructed according to molecular biology techniques well known in the art. See Brown T, “Gene Cloning” (Chapman & Hall, London, G B, 1995); Watson R, et al., “Recombinant DNA”, 2′ Ed. (Scientific American Books, New York, N.Y., US, 1992); Alberts B, et al., “Molecular Biology of the Cell” (Garland Publishing Inc., New York, N.Y., US, 2008); Innis M, et al., Eds., “PCR Protocols. A Guide to Methods and Applications” (Academic Press Inc., San Diego, Calif., US, 1990); Erlich H, Ed., “PCR Technology. Principles and Applications for DNA Amplification” (Stockton Press, New York, N.Y., US, 1989); Sambrook J, et al., “Molecular Cloning. A Laboratory Manual” (Cold Spring Harbor Laboratory Press, Cold Spring Harbor, N.Y., US, 1989); Bishop T, et al., “Nucleic Acid and Protein Sequence. A Practical Approach” (IRL Press, Oxford, G B, 1987); Reznikoff W, Ed., “Maximizing Gene Expression” (Butterworths Publishers, Stoneham, Mass., US, 1987); Davis L, et al., “Basic Methods in Molecular Biology” (Elsevier Science Publishing Co., New York, N.Y., US, 1986), Schleef M, Ed., “Plasmid for Therapy and Vaccination” (Wiley-VCH Verlag GmbH, Weinheim, Del., 2001).

For instance, HEK-293 cells (expressing E1 genes), a helper plasmid providing adenovirus function, a helper plasmid providing AAV rep genes from serotype 2 and cap genes from the desired serotype (e.g. AAV8) and, finally, the backbone plasmid with ITRs and the construct of interest may be employed. To generate an AAV vector expressing the Fc-fusion protein derivative of the invention, the cDNA of the Fc-fusion protein derivative may be cloned into an AAV backbone plasmid under the control of a ubiquitous (e.g. CAG) or a cell-specific promoter.

AAV vectors (viral vector particles) may be generated by helper virus-free transfection of HEK293 cells using three plasmids with modifications. See Matsushita T, et al., Gene Ther. 1998; 5:938-945 and Wright J, et al., Mol. Ther. 2005; 12:171-178. Cells may be cultured to 70% confluence in roller bottles (RB) (Corning Inc., Corning, N.Y., USA) in DMEM (Dulbeccos's Modified Eagle Medium) supplemented with 10% BFS (bovine fetal serum) and then co-transfected with: 1) a plasmid carrying the expression cassette flanked by the viral ITRs ‘described above); 2) a helper plasmid 33ucliying the AAV rep2 and the correspondent cap (capl and cap9 genes; and 3) a plasmid carrying the adenovirus helper functions. Vectors may then be purified by two consecutives cesium chloride gradients using either a standard protocol or an optimized protocol as previously described. See Ayuso E, et al., Gene Ther. 2010; 17:503-510. Vectors may be further dialyzed against PBS, filtered, titred by qPCR (quantitative polymerase chain reaction) and stored at −80° C. until use.

In another embodiment, the present invention relates to a host cell comprising a nucleic acid, expression cassette or vector of the invention. Host cells to be used according to the present invention can be of any cell type, including both eukaryotic cells and prokaryotic cells. Preferably, the cells include prokaryotic cells, yeast cells or mammalian cells. More preferably, the host cells are HEK-293 and CHO cells.

### 4. Pharmaceutical Compositions

In a further aspect, the present invention refers to a pharmaceutical composition containing at least one of the Fc-fusion protein derivatives, nucleic acids, vectors or host cells of the invention (hereinafter referred singly or jointly as “active agent(s) of the invention”) or a mixture thereof, formulated with a pharmaceutically acceptable carrier. Said pharmaceutical compositions are used for treating HIV or AIDS in a subject or preventing HIV infection in an33uclidean33d subject. In one embodiment, the compositions include a mixture of multiple (e.g. two or more) Fc-fusion protein derivatives, nucleic acids, vectors or host cells of the invention. In one embodiment of the invention, the composition includes a Fc-fusion protein derivative comprising at least one of sequences SEQ ID NO:13-25 or the nucleic acids, vectors or host cells expressing said Fc-fusion protein derivatives or a mixture thereof. Preferably, the composition includes a Fc-fusion protein derivative comprising at least one of sequences SEQ ID NO:19-25 or the nucleic acids, vectors or host cells expressing said Fc-fusion protein derivatives or a mixture thereof. More preferably, the composition includes a Fc-fusion protein derivative comprising at least one of sequences SEQ ID NO:20 or the nucleic acids, vectors or host cells expressing said Fc-fusion protein derivative or a mixture thereof. The preparation of pharmaceutical compositions comprising the Fc-fusion protein derivatives of the invention is known in the art. See McNally E, et al., Eds., “Protein Formulation and Delivery” (Marcel Dekker, Inc., New York, N.Y., USA, 2000), Hovgaard L, et al., Eds., “Pharmaceutical Formulation Development of Peptides and Proteins”, 2^{nd} Ed. (CRC Press, Boca Raton, Fla., USA, 2012) and Akers M, et al., Pharm Biotechnol. 2002; 14:47-127.

Preferably, the carrier is suitable for intravenous, intramuscular, subcutaneous, parenteral, spinal or epidermal administration (e.g. by injection or infusion). Depending on the route of administration, the active agent of the invention may be coated in a material to protect the agent from the action of conditions that may inactivate the agent.

In a further embodiment of the present invention, pharmaceutical compositions specifically suitable for gene therapy (“passive immunization”) are provided. Said pharmaceutical compositions comprise at least one of the nucleic acids or vectors of the invention or their mixture and are prepared according to methods known in the art. See André S, et al., J. Virol. 1998, 72:1497-1503; Mulligan M, Webber J, AIDS 1999; 13(Suppl A):S105-S112; O'Hagan D, et al., J. Virol. 2001; 75:9037-9043 and Rainczuk A, et al., Infect. Immun. 2004; 72:5565-5573. The particular vector backbone into which the nucleic acids of the invention are inserted is not important as long as said nucleic acid is adequately expressed in a subject. Examples of suitable vectors include, but are not limited to, viruses and plasmids. Preferably, an AAV vector is used when a viral vector is employed. Preferably, a pcDNA3.1 and pVAX1 (Invitrogen, Carlsbad, Calif., USA); DNA sequences available at the Invitrogen website thermofisher.com/uk/en/home/brands/34uclidean34, October 2015); pNGVL (National Gene Vector Laboratory, University of Michigan, Mich., USA); and p414cyc (ATCC accession number 87380) and p414GALS (ATCC accession number 87344) is used when a plasmidic vector is employed. More preferably, a pcDNA3.1 plasmid is utilized as plasmidic vector. Most preferably, the plasmidic vector is pM5A16T20 and pM7A16LLC34.

The design and applications of passive immunization are known in the art. See Donnelly J, et al., Annu. Rev. Immunol. 1997; 15:617-648; Robinson H, Pertmer T, Adv. Virus Res. 2000; 55:1-74; Gurunathan S, et al., Annu. Rev. Immunol. 2000; 18:927-974 (2000) and Ulmer J, Curr. Opin. Drug Discov. Devel. 2001; 4:192-197. Briefly, passive immunization within the context of the present invention is configured to direct the in vivo expression of a Fc-fusion protein derivative in a subject. See Ulmer J, et al., Science 1993; 259:1745-1749. Typically, the nucleic is cloned into a bacterial plasmid that is optimized for expression in eukaryotes and consists of the following: (i) an origin of replication for propagation in bacteria, usually an E. coli origin such as ColE1, (ii) an antibiotic resistance gene, usually kanamycin, for selection of the plasmid in bacteria, (iii) a strong promoter for optimal expression in mammalian cells like cytomegalovirus (CMV) or simian virus 40 (SV40), (iv) multiple cloning site downstream of the promoter for insertion of the gene of interest and (v) SV40 or bovine growth hormone (BGH) polyadenylation signal for stabilization of mRNA.

Still another object of the present invention is to deliver vectors utilizing non-pathogenic or attenuated bacterial strains harboring plasmids capable of expressing the Fc-fusion protein derivatives of the invention, such as, but not restricted to, Escherichia spp., Salmonella spp., Shigella spp., Mycobacterium spp. And Listeria spp.

The particular Escherichia strain employed is not critical to the present invention. Examples of Escherichia strains which can be employed in the present invention include Escherichia coli strains DH5a, HB 101, HS-4, 4608-58, 1184-68, 53638-C-17, 13-80, and 6-81, enterotoxigenic E. coli, enteropathogenic E. coli and enterohemorrhagic E. coli. See Sambrook, 1989, supra; Sansonetti P, et al., Ann. Microbiol. 1982; 132A:351-355); Evans D, et al., Infect. Immun. 1975; 12:656-667; Donnenberg S, et al., J. Infect. Dis. 1994; 169:831-838 and McKee M, O'Brien A, Infect. Immun. 1995; 63:2070-2074.

The particular Salmonella strain employed Is not critical to the present invention. Examples of Salmonella strains that can be employed in the present invention include S. typhi (ATCC accession number 7251), S. typhimurium (ATCC accession number 13311), S. galinarum (ATCC accession number 9184), S. enteriditis (ATCC accession number 4931), S. typhimurium (ATCC accession number 6994), S. typhi aroC, aroD double mutant (Hone D, et al., Vaccine 1991; 9:810-816) and S. typhimurium aroA mutant (Mastroeni D, et al., Micro. Pathol. 1992; 13:477-491).

The particular Shigella strain employed is not critical to the present invention. Examples of Shigella strains that can be employed in the present invention include S. flexneri (ATCC accession number 29903), S. flexneri CVD1203 (ATCC accession number 55556), S. flexneri 15D (Sizemore D, et al., Vaccine 1997; 15:804-807; Sizemore D, et al., Science 1995, 270:299-302), S. sonnei (ATCC accession number 29930) and S. dysenteriae (ATCC accession number 13313).

The particular Mycobacterium strain employed is not critical to the present invention. Examples of Mycobacterium strains that could be employed in the present invention include M. tuberculosis CDC1551 strain (Griffith T, et al., Am. J. Respir. Crit. Care Med. 1995; 152:808-811), M. tuberculosis Beijing strain (van Soolingen D, et al., J. Clin. Microbiol. 1995; 33:3234-3238), M. tuberculosis H37Rv strain (ATCC accession number 25618), M. tuberculosis pantothenate auxotroph strain (Sambandamurthy V, Nat. Med. 2002; 8:1171-1174, M. tuberculosis rpoV mutant strain (Collins D, et al., Proc. Natl. Acad. Sci USA. 1995; 92: 8036, M. tuberculosis leucine auxotroph strain (Hondalus M, et al., Infect. Immun. 2000; 68(5):2888-2898), BCG Danish strain (ATCC accession number 35733), BCG Japanese strain (ATCC accession number 35737), BCG, Chicago strain (ATCC accession number 27289), BCG Copenhagen strain (ATCC No. 27290), BCG Pasteur strain (ATCC accession number 35734), BCG Glaxo strain (ATCC accession number 35741), BCG Connaught strain (ATCC accession number 35745) and BCG Montreal (ATCC accession number 35746).

The particular Listeria strain employed is not critical to the present invention. Examples of Listeria monocytogenes strains which can be employed in the present invention include, but are not restricted to, L. monocytogenes strain 10403S (Stevens R, et al., J. Virol. 2004; 78:8210-8218), L. ivanovii and L. seeligeri strains (Haas A, et al., Biochim. Biophys. Acta. 1992; 1130:81-84) or mutant L. monocytogenes strains such as (i) actA plcB double mutant (Peters C, et al., FEMS Immunol. Med. Microbiol. 2003; 35:243-253 and Angelakopoulos H, et al., Infect. Immun. 2002; 70:3592-3601) or (ii) dal dat double mutant for alanine racemase gene and D-amino acid aminotransferase gene (Thompson R, et al., Infect. Immun. 1998; 66:3552-3561).

Methods for delivering vectors using bacterial vehicles are well known in the art. See U.S. Pat. Nos. 6,500,419, 5,877,159 and 5,824,538; Shata M, et al., Mol. Med. Today 2000; 6:66-71; Hone D, Shata M, J. Virol. 2001; 75:9665-9670; Shata M, et al., Vaccine 2001; 20:623-629; Rapp U and Kaufmann S, Int. Immunol. 2004, 16:597-605; Dietrich G, et al., Curr. Opin. Mol. Ther. 2003; 5:10-19 and Gentschev I, et al., J. Biotechnol. 2000; 83:19-26. The type of plasmid delivered by said bacterial vehicles for expressing the Fc-fusion protein derivatives of the invention is not critical.

In an additional embodiment, the use of an AAV vector for delivering the nucleic acids of the invention is also provided.

The pharmaceutical compositions of the present invention can be administered by a variety of methods known in the art. As will be appreciated by the skilled artisan, the route or mode of administration will vary depending upon the desired results. The active agents of the invention can be prepared with carriers that will protect the agent against rapid release, such as a controlled release formulation, including implants, transdermal patches and microencapsulated delivery systems. Biodegradable, biocompatible polymers can be used, such as ethylene vinyl acetate, polyanhydrides, polyglycolic acid, collagen, polyorthoesters and polylactic acid. Many methods for the preparation of such formulations are generally known to in the art. See Robinson J, et al., Eds., “Sustained and Controlled Release Drug Delivery Systems” (Marcel Dekker, Inc., New York, N.Y., USA, 1978).

To administer an active agent of the invention by certain routes of administration, it may be necessary to coat the agent with, or co-administer the agent with, a material to prevent its inactivation or to ensure its proper distribution in vivo. For example, the agent may be administered to a subject in an appropriate carrier (e.g. liposome) or a diluent. Pharmaceutically acceptable diluents include, but are not limited to, saline and aqueous buffer solutions. Liposomes include water-in-oil-in-water CGF emulsions as well as conventional liposomes. See Strejan G, et al., J. Neuroimmunol. 1984; 7:27-41. Many methods of manufacturing liposomes are known in the art. See U.S. Pat. Nos. 4,522,811, 5,374,548 and 5,399,331. The liposomes may comprise one or more moieties which are selectively transported into specific cells or organs and thus enhance targeted drug delivery. Exemplary targeting moieties include folate or biotin, mannosides and surfactant protein A receptor. In one embodiment of the invention, the active agents of the invention are formulated in liposomes; in a more preferred embodiment, the liposomes include a targeting moiety. In a most preferred embodiment, the active agents in the liposomes are delivered by bolus injection.

Pharmaceutically acceptable carriers include sterile aqueous solutions or dispersions and sterile powders for the extemporaneous preparation of sterile injectable solutions or dispersions. The use of such media in the preparation of the pharmaceutical compositions of the invention is contemplated herein in so far as their use is not incompatible with the active agents of the invention. Supplementary active compounds can also be incorporated into the pharmaceutical compositions.

Pharmaceutical compositions are typically sterile and stable under the conditions of manufacture and storage. The composition can be formulated as a solution, microemulsion, liposome or other ordered structure suitable to active agent concentration. The carrier can be a solvent or dispersion medium containing, for example, water, ethanol, polyol (e.g. glycerol, propylene glycol, liquid polyethylene glycol) or suitable mixtures thereof. The proper fluidity can be maintained, for example, by using a coating such as lecithin, by reducing the deviation in particle size and by using surfactants. In many cases, it will be preferable to include isotonic agents, such as, for example, sugars, polyalcohols (e.g. mannitol, sorbitol) or sodium chloride in the composition. Prolonged absorption of the injectable compositions can be brought about by including in the composition compounds that delay absorption (e.g. monostearate salts, gelatin).

Sterile injectable solutions can be prepared by incorporating the active agent of the invention in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (i.e. lyophilization) that yield a powder of the active ingredient plus any additional desired ingredient from a previously sterile-filtered solution thereof.

Dosage regimens are adjusted to provide the optimum desired response (e.g. a therapeutic response). For example, a single bolus may be administered, several divided doses may be administered over time or the dose may be proportionally reduced or increased, as indicated by the exigencies of the therapeutic situation. For example, the Fc-fusion protein derivatives of the invention may be administered once or twice weekly by subcutaneous injection or once or twice monthly by subcutaneous injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active agent calculated to produce the desired therapeutic effect in association with the required pharmaceutical carrier. The specification for the dosage unit forms of the invention are dictated by and directly dependent on the unique characteristics of the active agent and the particular therapeutic effect to be achieved.

Examples of pharmaceutically-acceptable antioxidants include, but are not limited to, water soluble antioxidants (e.g. ascorbic acid, cysteine hydrochloride, sodium bisulfate, sodium metabisulfite, sodium sulfite), oil-soluble antioxidants (e.g. ascorbyl palmitate, butylated hydroxyanisole (BHA), butylated hydroxytoluene (BHT), lecithin, propyl gallate, alpha-tocopherol) and metal chelating agents (e.g. citric acid, ethylenediamine tetraacetic acid (EDTA), sorbitol, tartaric acid, phosphoric acid). The formulations of the pharmaceutical compositions of the invention include those suitable for oral, nasal, topical (e.g. buccal and sublingual), rectal, vaginal or parenteral administration. The formulations may conveniently be presented in unit dosage form and may be prepared by any methods known in the art. The amount of active agent which can be combined with a carrier material to produce a single dosage form will vary depending upon the subject being treated and the particular mode of administration. The amount of active agent which can be combined with a carrier material to produce a single dosage form will generally be that amount of the composition which produces a therapeutic effect. Generally, this amount will range from about 0.001% to about 90% of active agent, preferably from about 0.005% to about 70% and, most preferably, from about 0.01% to about 30%.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active agent of th39uclidntion may be mixed under sterile conditions with a pharmaceutically acceptable carrier and with any preservatives, buffers or propellants which may be required.

The pharmaceutical compositions of the invention may also contain adjuvants such as preservatives, wetting agents, emulsifying agents and dispersing agents. Prevention of presence of microorganisms may be ensured both by sterilization procedures and by the inclusion of various antibacterial and antifungal agents (e.g. paraben, chlorobutanol, phenol sorbic acid). It may also be desirable to include isotonic agents (e.g. sugars, sodium chloride) into the compositions.

Actual dosage levels of the active agents in the pharmaceutical compositions of the present invention may be varied for attaining the desired therapeutic response in a subject. The selected dosage level will depend upon a variety of pharmacokinetic factors including the activity of the particular agent of the invention employed, its amount, the route of administration, the time of administration, the rate of excretion or expression of the particular active agent employed, the duration of the treatment, other drugs, compounds or materials used in combination with the particular pharmaceutical compositions employed, the age, sex, weight, condition, general health and prior medical history of the subject being treated and other similar factors known in the medical arts. A physician or veterinarian having ordinary skill in the art can readily determine and prescribe the therapeutically effective amount of the active agent(s) required. For example, the physician or veterinarian could start doses of the active agents of the invention employed in the pharmaceutical composition at levels lower than that required for achieving the desired therapeutic effect and gradually increase the dosage until the desired effect is achieved. In general, a suitable daily dose of a composition of the invention will be that amount of the active agent which is the lowest dose effective to produce a therapeutic effect. Such an effective dose will generally depend upon the factors described above. It is preferred that administration be parenteral, more preferably intravenous, intramuscular, intraperitoneal or subcutaneous. If desired, the effective daily dose of a pharmaceutical composition may be administered as two, three, four, five, six or more sub-doses applied separately at appropriate intervals throughout the day, optionally, in unit dosage forms. While it is possible for an active agent of the invention to be administered alone, it is preferable to administer said agent as a pharmaceutical composition.

The pharmaceutical compositions of the invention can be administered with medical devices known in the art. For example, in a preferred embodiment, the pharmaceutical composition of the invention can be administered with a needleless hypodermic injection device. See U.S. Pat. Nos. 5,399,163, 5,383,851, 5,312,335, 5,064,413, 4,941,880, 4,790,824 or U.S. Pat. No. 4,596,556. Examples of well-known implants and modules useful in the present invention include, but are not limited to, infusion pumps for dispensing medications at different rates (e.g. U.S. Pat. No. 4,447,233 (non-implantable, controlled rate), U.S. Pat. No. 4,447,224 (implantable, variable rate), U.S. Pat. No. 4,487,603 (implantable, controlled rate)), devices for administering medicaments through the skin (e.g. U.S. Pat. No. 4,486,194) and osmotic drug delivery systems (e.g. U.S. Pat. Nos. 4,439,196 and 4,475,196). Many other such implants, delivery systems and modules are known to those skilled in the art.

The pharmaceutical compositions of the invention must be sterile and fluid to the extent that the composition is deliverable by syringe. In addition to water, the carrier can be an isotonic buffered saline solution, ethanol, polyol (e.g. glycerol, propylene glycol, liquid40uclidean40nee glycol) and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols (e.g. mannitol, sorbitol) and sodium chloride in the composition. Long-term absorption of the injectable compositions can be brought about by including an agent which delays absorption (e.g. aluminum monostearate, gelatin) in the composition.

### 5. Methods of Treatment and Prevention

In another aspect, the invention is directed to a method for either treating or preventing HIV infection or AIDS in a subject which comprises the administration to said subject of at least one of the Fc-fusion protein derivatives, nucleic acids, vectors, host cells or pharmaceutical compositions of the invention, or a mixture thereof. The beneficial treatment or preventive effects of the active agents and pharmaceutical compositions of the invention in relation to HIV infection or AIDS symptoms include, for example, preventing or delaying initial infection of a subject exposed to HIV, reducing viral burden in a subject infected with HIV, prolonging the asymptomatic phase of HIV infection, maintaining low viral loads in HIV infected subjects whose virus levels have been lowered via anti-retroviral therapy (AT), increasing levels of CD4 T cells or lessening the decrease in CD4 T cells, both HIV-1 specific and non-specific, in drugle subjects and in subjects treated with AT, increasing overall health or quality of life in a subject with AIDS and prolonging the life expectancy of a subject with AIDS. A physician or veterinarian can compare the effect of the treatment wi4luclidesubject's condition prior to treatment, or with the expected condition of an untreated subject, to determine whether the treatment is effective in inhibiting AIDS. In a preferred embodiment, the active agents and pharmaceutical compositions of the invention are used for the prevention of HIV infection or AIDS. In another preferred embodiment, the active agents and pharmaceutical compositions of the invention are used for the treatment of HIV infection or AIDS.

The active agents and pharmaceutical compositions of the invention may be useful in the treatment of HIV infection or AIDS. While all subjects that can be afflicted with HIV or their equivalents can be treated in this manner (e.g. chimpanzees, macaques, baboons or humans), the active agents and pharmaceutical compositions of the invention are directed particularly to their therapeutic uses in humans. Often, more than one administration may be required to bring about the desired therapeutic effect; the exact protocol (dosage and frequency) can be established by standard clinical procedures.

The present invention further relates to reducing or eliminating the symptoms associated with HIV infection or AIDS. These include symptoms associated with the minor symptomatic phase of HIV infection, including, for example, shingles, skin rash and nail infections, mouth sores, recurrent nose and throat infection and weight loss. In addition, further symptoms associated with the major symptomatic phase of HIV infection, include, for instance, oral and vaginal thrush (Candida), persistent diarrhea, weight loss, persistent cough and reactivated tuberculosis or recurrent herpes infections, such as cold sores (herpes simplex). Other symptoms of full-blown AIDS which can be treated in accordance with the present invention include, for instance, diarrhea, nausea and vomiting, thrush and mouth sores, persistent, recurrent vaginal infections and cervical cancer, persistent generalized lymphadenopathy (PGL), severe skin infections, warts and ringworm, respiratory infections, pneumonia, especially Pneumocystis carinii pneumonia (PCP), herpes zoster (or shingles), nervous system problems, such as pains, numbness or “pins and needles” in the hands and feet, neurological abnormalities, ‘aposi's sarcoma, lymphoma, tuberculosis or other similar opportunistic infections.

In another preferred embodiment, the active agents or pharmaceutical compositions of the invention are administered to an HIV-infected subject or a subject exposed to HIV in combination with at least one therapeutic agent. Preferably, the therapeutic agent is indicated commonly for the prevention or treatment of HIV or AIDS. Suitable therapeutic agents include, but are not limited to, drugs forming part of current antiretroviral therapy (AT) and highly active antiretroviral therapy (HAART) protocols such as non-nucleoside reverse transcriptase inhibitor (e.g. efavirenz, nevirapine, delavirdine, etravirine, rilpivirine), nucleoside analogue reverse transcriptase inhibitors (e.g. zidovudine, tenofovir, lamivudine, emtricitabine) and protease inhibitors (e.g. saquinavir, ritonavir, indinavir, nelfinavir, amprenavir), referred hereinafter independently or jointly as “HIV antiretroviral(s)”. In one version of this embodiment, at least one active agent or pharmaceutical composition of the invention and at least one HIV antiretroviral are administered to the subject together at the same time. In another version, at least one active agent or pharmaceutical composition of the invention is administered before any HIV antiretroviral is applied to the subject. In yet another version, at least one active agent or pharmaceutical composition of the invention is administered after a HIV antiretroviral has been applied to the subject, such as, for example, after the interruption of an AT or HAART protocol.

Additionally, the Fc-fusion protein derivatives of the invention can be also administered with therapeutic agents that may induce the expression of HIV gp120 on the surface of latently infected cells, thus allowing their quick removal by NK cells. See Siliciano J, et al, J Allergy Clin Immunol. 2014; 134(1):12-19.

### 6. Neutralization and Detection Methods

In an additional aspect, the present invention relates to a method of inactivating HIV which comprises the step of contacting the virus with at least one Fc-fusion protein derivative of the invention. Preferably, the method is carried out over a sample containing HIV or suspected of containing HIV. The method may be conducted under conditions that favor the coupling of the Fc-fusion protein derivative to HIV as described in the art. See Lu L, et al., Retrovirology 2012; 9(104), 1-14.

In a further aspect, the present invention relates to a method of detecting a molecule or a fragment thereof (e.g. HIV, gp120) that attaches to the D1 and D2 domains of the human CD4 receptor in a sample which comprises the steps of (a) contacting the sample with a Fc-fusion protein derivative of the invention and (b) determining whether the Fc-fusion protein derivative specifically binds to a molecule in the sample. The sample may be a biological sample including, but not limited to, blood, serum, urine, tissue or other biological material from non-infected, infected or potentially infected subjects (e.g. subjects sustaining periodic or intermittent HIV exposure). The sample may also be non-biological (e.g. obtained from water, beverages). Preferably, the molecule is HIV and, more preferably, the gp120 viral envelope protein of HIV.

In a preferred embodiment of this aspect, the present invention relates to a method of detecting HIV in a sample which comprises the steps of (a) contacting the sample with a Fc-fusion protein derivative of the invention and (b) determining whether the Fc-fusion protein derivative specifically binds to a HIV molecule in the sample. Preferably, the sample is a plasma sample or a serum sample.

A sample may be first manipulated to make it more suitable for the method of detection. In a preferred embodiment, the contact between the sample and the Fc-fusion protein derivative is prolonged (i.e. an incubation under conditions suitable for the stability of the sample and the Fc-fusion protein derivative). The conditions during the contacting step can be determined in a routine manner by the skilled artisan. Suitable buffers that can be used in the contacting step include physiological buffers that do not interfere with the assay to be performed. For example, a Tris or a triethanolamine (TEA) buffer can be employed. The pH of the buffer (and resulting lysis reagent including the buffer solution) can range from about 2.0 to about 10.0, optionally from about 4.0 to about 9.0, preferably from about 7.0 to about 8.5 and even more preferably from about 7.5 to about 8.0, or, about 7.0, about 7.5, about 8.0, or about 8.5. Exemplary “contacting” conditions may comprise incubation for 15 minutes to 4 hours (e.g. 1 hr at 4° C., 37° C. or at room temperature). However, these may be varied as appropriate according to, for example, the nature of the interacting binding partners. The sample may optionally be subjected to gentle rocking, mixing or rotation. In addition, other appropriate reagents such as blocking agents to reduce non-specific binding may be added. For example, 1-4% BSA or other suitable blocking agent (e.g. milk) may be used. The contacting conditions can be varied and adapted depending on the aim of the detection method. For example, if the incubation temperature is, for example, room temperature or 37° C., this may increase the possibility of identifying binders which are stable under these conditions (e.g. stable under conditions found in the human body).

Preferably, the Fc-fusion protein derivatives of the invention are contacted with the sample under conditions which allow the formation of a complex between the Fc-fusion protein derivative and a molecule or fragment thereof present in the sample. The formation of a complex indicating, for example, the presence of HIV in the sample is then detected and measured by suitable means. These means of detection and measurement depend on the nature of the binding partners and include, but are not limited to, homogeneous and heterogeneous binding assays such as, for example, radio-immunoassays (RIA), ELISA, immunofluorescence, immunohistochemistry, flow cytometry (e.g. FACS), BIACORE and Western blot analyses. Preferred assay techniques, especially for large-scale analysis of subject sera and blood and blood-derived products, are flow cytometry, ELISA and Western blot techniques.

In a preferred embodiment, the measuring is performed by flow cytometry (e.g. FACS). As used herein, the term “flow cytometry” refers to an assay in which the proportion of a material in a sample is determined by labeling the material (e.g. by binding a labeled antibody to the material), causing a fluid stream containing the material to pass through a beam of light, separating the light emitted from the sample into constituent wavelengths by a series of filters and mirrors and detecting the light. Flow cytometry permits sensitive detection and rapid quantification of some features of single cells, such as relative size complexity and endogenous fluorescence, as well as the quantitative analysis of any cellular compound that can be labeled with a fluorochrome. See Melamed M, et al., “Flow Cytometry and Cell Sorting”, 2nd Ed. (Wiley-Liss, New York, N.Y., USA, 1990). One of the main advantages of flow cytometry is the rapid quantification of analytes on a large number of particles or cells. Generally, the fluorochromes selected for use as detectable markers are selected based on their ability to fluoresce when excited by light with the wavelength used by the laser. When the fluorochrome is excited by the laser beam, it emits light which is then assessed by the photomultiplier tubes of the flow cytometer. This technique is capable of analyzing 10,000 cells/particles within 1 to 2 minutes. Flow cytometers have filters to detect the emittance from various fluorochromes which fluoresce at different wavelengths and allow for four or more different fluorochromes to be used as detectable markers, which means currently at least 4 different molecules may be detected simultaneously. These methods and apparatus for analyzing sample are commercially available and are well known in the art (e.g. FACSCalibur Flow Cytometer; BD Biosciences Corp., Franklin Lakes, N.J., USA).

In a preferred embodiment, said measuring comprises the analysis of the sample, preferably by flow cytometry using a reporter capable of binding to the Fc-fusion protein derivative, preferably, to the Fc region of said Fc-fusion protein derivative. Preferably, the reporter comprises a detectable moiety and, more preferably, it is a Fc-specific secondary antibody coupled to a detectable moiety.

Useful detectable moieties include fluorophores. By “fluorophore” (or “fluorochrome” or “chromophore”) it is understood a fluorescent compound that can re-emit light upon light excitation. Fluorophores that can be used include biological (e.g. proteins) and chemical fluorophores. Exemplary biological fluorophores comprise T-sapphire, Cerulean, mCFPm, CyPet, EGFP, PA-EGFP, Emerald, EYFP, Venus, mCitrine, mKO, mOrange, DSred, JRed, mStrawberry, mCherry, PA-mCherry, mRuby, Tomato, mPlum, mKate, mKatushka, Kaede, Halotag, and superecliptic fluorine. Exemplary chemical fluorophores comprise Alexafluor, Rhodamine, BODIPY, Tetramethylrhodamine, Cyanin dyes, Fluorescein, Quantum dots, IR dyes, FM dyes, ATTO dye. A secondary antibody can also be labeled with enzymes that are useful for detection, such as, for example, horseradish peroxidase, β-galactosidase, luciferase, alkaline phosphatase or glucose oxidase. When an antibody is labeled with a detectable enzyme, it can be detected by adding additional reagents that the enzyme uses to catalyze a reaction product that can be discerned. For example, when the agent horseradish peroxidase is present, the addition of hydrogen peroxide and diaminobenzidine leads to a colored reaction product, which is visually detectable. An antibody may also be labeled with biotin and detected through indirect measurement of avidin or streptavidin binding. It should be noted that the avidin itself can be labeled with an enzyme or a fluorescent label.

Preferably, the Fc secondary antibody employed is specific for the species from which the primary antibody is derived (e.g. human). In one embodiment, said Fc-specific secondary antibody is selected from the group consisting of IgA (e.g. IgA1, IgA2), IgD, IgE, IgG (e.g. IgG1, IgG2, IgG3, IgG4) and IgM. Preferably, the secondary antibody is IgG and, more preferably, IgG1. Said Fc-specific secondary antibody can be any vertebrate antibody, preferably any mammal antibody and, more preferably, any non-human antibody (e.g. a rabbit, mouse, rat, goat, horse, sheep or donkey antibody).

For use as reagents in the aforementioned assays, the Fc-fusion protein derivatives of the invention may be conveniently bonded to the inside surface of microtiter wells. The Fc-fusion protein derivatives of the invention may be directly bonded to the microtiter well. However, maximum binding of the Fc-fusion protein derivatives to the wells might be accomplished by pre-treating the wells with polylysine prior to the addition of the Fc-fusion protein derivatives. Furthermore, the antibody derivatives of the invention may be covalently attached to the wells by means known in the art. Generally, the Fc-fusion protein derivatives of the invention are used between 0.01 to 100 μg/mL for coating, although higher as well as lower amounts may also be used. Samples are then added to the wells coated with the Fc-fusion protein derivatives of the invention.

### 7. Kits

In a further aspect, the present invention refers to kits comprising at least one of the antibody derivatives, nucleic acids, vectors, host cells, pharmaceutical compositions or combinations of the invention or mixtures thereof. The components of the kits of the invention may be optionally packed in suitable containers and be labeled for the detection, inactivation, diagnosis, prevention or treatment of HIV or AIDS or their related condition46uclideanmponents of the kits may be stored in unit or multi-dose containers as an aqueous, preferably sterile, solution or as a lyophilized, preferably sterile, formulation for reconstitution. The containers may be formed from a variety of materials such as glass or plastic and may have a sterile access port (e.g. the container may be an intravenous solution bag or a vial having a stopper pierceable by a hypodermic injection needle). The kits may further comprise more containers comprising a pharmaceutically acceptable carrier. They may further include other materials desirable from a commercial and user standpoint, including, but not limited to, buffers, diluents, filters, needles, syringes, culture medium for one or more of the suitable host cells or other active agents. The kits can contain instructions customarily included in commercial packages of diagnostic and therapeutic products that contain information, for example, about the indications, usage, dosage, manufacture, administration, contraindications or warnings concerning the use of such diagnostic and therapeutic products.

All publications mentioned herein are incorporated in their entirety by reference. Having now generally described the invention, the same will be more readily understood through reference to the following examples, which are provided by way of illustration and are not intended to be limiting of the present invention, unless specified.

### General Procedures

1. Construction of Fc Fusion Protein Derivatives

The two extracellular domains of human CD4 molecule (D1 and D2) were joined to the Fc portion of the human IgG1 comprising G236A, S239D, A330L and I332E point mutations, which include the hinge, CH2 and CH3 domains, to yield a modified CD4-IgG1 molecule. Based on the modified CD4-IgG1 scaffold, Fc-fusion protein derivatives were designed with the following characteristics:
(a) M428L and N434S point mutations were made to the Fc chain to extend the activity of the Fc-fusion protein derivatives.
(b) K322A point mutation were made to modulate complement activation.
(c) F243L and R292P point mutations were made to the Fc chain to improve the production of the Fc-fusion protein derivatives.
(d) At least one or more of a Y300L, P396L, S298A, E333A or K334A point mutations were made to the Fc chain to further improve the production of the Fc-fusion protein derivatives.
(e) Addition of the N-terminal extracellular sequence of human CCR5 (SEQ ID NO:5) at the C-terminus of the Fc chain.
(f) Addition of the T-20 (SEQ ID NO:10), C34 (SEQ ID NO:11) or EHO (SEQ ID NO:12) sequence at the C-terminus of the Fc chain.
(g) Sequential addition of (i) the CCR5 sequence (SEQ ID NO:5), (ii) the T-20 (SEQ ID NO:10), C34 (SEQ ID NO:11) or EHO (SEQ ID NO:12) sequence and (iii) a variable number of a flexible link (GGGGS) (SEQ ID NO:4) and/or SEQ ID NO: 6-9 at the C-terminus of the Fc chain.

All the polynucleotides expressing the Fc-fusion protein derivatives of the invention were synthetized using the GeneArt process and the pcDNA3.1 and pcDNA3.4 expression plasmids.

Plasmids were reconstituted with 10 mM Tris buffer pH8 at 0.5 μg/μL. One Shot TOP10 chemically competent E. coli (Life Technologies Corp., Carlsbad, Calif., USA) were transformed using 1 μL of plasmid and following the manufactured instruction. In brief, 1 μL of plasmid was added to a vial of bacteria and incubated on ice for 15 minutes. After that, the tube was incubated at 42° C. for 30 seconds and immediately left on ice for two minutes. Bacteria were resuspended in 250 μL of SOC medium (Life Technologies Corp., Carlsbad, Calif., US) and incubated for 1 hour at 37° C. and 225 rpm in an Innova 4000 incubator shaker (New Brunswick Scientific Co., Inc., Enfield, Conn., USA). After that, 100 μL of a 1/100 dilution of the cell culture were spread onto ampicillin selection (100 μg/mL) LB-Agar plates. Plates were incubated at 37° C. from 16-24 hours into a Heraeus incubator (Thermo Fisher Scientific, Waltham, Mass., USA). One colony was isolated and inoculated into 5 mL of ampicillin (100 μg/mL) selection LB medium and incubated for 8 hours at 37° C. and 225 rpm in an Innova 4000 incubator shaker (New Brunswick Scientific Co., Inc., Enfield, Conn., USA). After that, 500 mL of ampicillin (100 μg/mL) selection LB medium was inoculated with 500 μL of the former described culture and incubated at 37° C. and 225 rpm for 16 hours as described previously. Bacteria were harvested by centrifugation at 3000×g for 45 minutes at room temperature in an Eppendorf centrifuge 5810R (Thermo Fisher Scientific, Waltham, Mass., USA) and plasmids were isolated using the Qiagen Plasmid Maxi Kit (Qiagen NV, Venlo, NL) and following the manufacturer's instructions. Purified plasmids were quantified by spectrophotometry using a nanodrop 1000 instrument (Thermo Fisher Scientific, Waltham, Mass., USA).

2. Protein Production, Quantification, and Purification

HEK-293 cells were transfected with the plasmids encoding for the different Fc-fusion protein derivatives of the invention using Calphos transfection kit (Clontech®, Takara Bio Inc., Otsu, J P) following the manufacturer's instructions. After 48 hours, the supernatant was collected, clarified by filtration through a 0.45 μm filter (EMD Millipore, Merck KGaA, Darmstadt, DE) and stored at −20° C. until use.

The Fc-fusion protein derivatives were quantified by ELISA. In brief, Maxisorp 96-F plates (Nunc, Thermo Fisher Scientific, Waltham, Mass., USA) were incubated overnight at 4° C. with 100 μL/well of a F(ab)2 goat anti-human IgG (Fc-specific) antibody (Jackson ImmunoResearch Labs, Inc., West Grove, Pa., USA) at 1 μg/mL in PBS. After blocking with PBS/10% FBS/0.05% tween20 and washing, serial dilutions of culture supernatant (containing the recombinant protein) in a blocking buffer were added to the plate (100 μL/well) and incubated overnight at 4° C. The plates were washed again and a secondary antibody HRP-F(ab)2 Goat anti-human IgG (Fc-specific) (Jackson ImmunoResearch Labs, Inc., West Grove, Pa., USA) at 1/10000 dilution in blocking buffer was added (100 μL/well) and incubated at room temperature for one hour. Plates were washed and the bound antibodies were detected using OPD substrate and the reaction stopped adding 4N H₂SO₄. The product was measured at 492 nm in an ELISA plate reader.

Proteins were purified using prot49uclideanharose (GE Healthcare, Inc., Stamford, Conn., USA) column. Proteins were produced by transient transfection using serum free medium, as indicated above. Supernatants were harvested, centrifuged at 3000×g for 10 minutes and filtered at 0.45 μm to remove cell debris. Clarified supernatant was added to previously washed prot49uclideanharose beads and incubated overnight at 4° C. with end to end rotation. Protein A was washed with Tris buffer saline (TB S) and the bound protein eluted with 4M MgCl₂. Alternatively, proteins were purified using CaptureSelect FcXL Affinity Matrix (Thermo Fisher Scientific, Waltham, Mass., USA) columns and eluted with glycine buffer pH=3.5. Purified proteins were dialyzed against PBS, concentrated by ultrafiltration, quantified by ELISA or spectrophotometry and stored at −80° C. until use.

3. Neutralization Assays

HIV-1 isolates NL4-3, BaL, AC10, SVBP6, SVBP8, SVBP11, SVBP12, SVBP14, SVBP15, SVBP17, SVBP18 and SVBP19 were generated as pseudoviruses using Env expression plasmids and the pSG3 vector. See Sanchez-Palomino S, et al., Vaccine 2011; 29:5250-5259. Cell-free virus neutralization by the Fc-fusion protein derivatives was tested by a standard TZM-bl based assay. See Li, 2005, supra. Briefly, in a 96-well culture plate, 100 μL of previously diluted Fc-fusion protein derivatives were preincubated with 50 μL of pseudovirus stock, using 200 TCID₅₀ (tissue culture infectious doses) at 37° C. for one hour. Then, 100 μL containing 10,000 TZM-bl luciferase-reporter target cells per well were added. Plates were cultured at 37° C. and 5% CO₂ for 48 hours. Serial dilutions of the Fc-fusion protein derivatives were tested, from 1000 to 0.1 ng/mL. TZM-bl reporter cells were treated with dextran (Sigma-Aldrich, Saint Louis, Mo., USA) to enhance infectivity. A luciferase substrate, Britelite Plus (PerkinElmer, Inc., Waltham, Mass., USA), was used for the readout. Non-linear fit of neutralization data was calculated using normalized values fitted to a one site inhibition curve with variable Hill slope. All statistical analyses and non-linear fitting were performed using the GraphPad Prism v5.0 software.

4. ADCC Assays

To evaluate the ability of the different Fc-fusion protein derivatives to activate NK mediated destruction of HIV infected cells by NK Cells, an ADCC assay was conducted according to Alpert M, et al., J. Virol. 2012, 86:12039-12052. Briefly, the NK cell line KHYG-1 CD16+ was employed as the source of effector cells and the CEM.NKR.CCR5+ Luc cell line was utilized as the source of target cells. Five days prior to the assay, the target cells were infected with a highly infectious BaL isolate stock and cultured in R10 medium (RPMI supplemented with 10% fetal calf serum) at 37° C. To set up the assay, 10⁴ target cells (>40% of them productively infected) were cultured with 10⁵ effector cells in R10 medium supplemented with 10 U/ml of recombinant IL-2 in the presence of different concentrations of the Fc-fusion protein derivatives or antibody-based molecules in a total volume of 200 μL in U bottom 96 well plates. After 8 hours of incubation at 37° C., cells were resuspended and 150 μL of cells suspension was mixed with 50 μL of luciferase substrate, Britelite Plus (PerkinElmer, Inc., Waltham, Mass., USA). Luciferase units were used for the readout. Since target cells express luciferase upon HIV infection, the reduction of luciferase activity is a direct measure of antibody- and NK mediated-cell killing.

5. Binding to Fc Receptors (ELISA Assays)

To evaluate the binding affinity of the Fc-fusion protein derivatives to human Fc receptors, different ELISA assays using recombinant soluble human CD64, CD32, CD16 and FcRn proteins were conducted (R&D Systems, Bio-Techne Ltd, Abingdon, GB). In brief, Maxisorp 96-F plates (Nunc, Thermo Fisher Scientific, Waltham, Mass., USA) were incubated overnight at 4° C. with 100 μL/well (1 μg/mL in PBS) of mouse anti-6×His clone HIS.H8 (Life Technologies, Thermo Fisher Scientific, Waltham, Mass., USA). After blocking with PBS/1% BSA/0.05% tween20 and washing, serial dilutions of Fc-fusion protein immunocomplexes dissolved in a blocking buffer were added to the plate (100 μL/well) and incubated overnight at 4° C. Immunocomplexes were prepared by incubation of Fc-fusion proteins (1 μg), biotin conjugated mouse-anti-human CD4 (1.4 μg) (clone SK3, Biolegend) and streptavidin (0.125 μg) (Jackson ImmunoResearch Labs, Inc., West Grove, Pa., USA) for two hours at room temperature. The plates were washed again and a secondary antibody HRP-F(ab)2 Goat anti-human IgG (Fc-specific) (Jackson ImmunoResearch Labs, Inc., West Grove, Pa., USA) at 1/10000 dilution in blocking buffer was added (100 μL/well) and incubated at room temperature for one hour. Plates were washed and the bound antibodies were detected using OPD substrate and the reaction stopped adding 4N H₂SO₄. The product was measured at 492 nm in an ELISA plate reader.

6. Binding to C1q (ELISA Assays)

To evaluate the binding affinity of the Fc-fusion protein derivatives to human C1q an ELISA assay using soluble human C1q were conducted (Bio-Rad Labs, Inc., Hercules, Calif., USA). In brief, Maxisorp 96-F plates (Nunc, Thermo Fisher Scientific, Waltham, Mass., USA) were incubated overnight at 4° C. with 100 μL/well (1 μg/mL in PBS) of human C1q. After blocking with PBS/1% BSA/0.05% tween20 and washing, serial dilutions of Fc-fusion protein were added and incu5luclideanrnigth at 4° C. The plates were washed again and a secondary antibody HRP-F(ab)2 Goat anti-human IgG (Fc-specific) (Jackson ImmunoResearch Labs, Inc., West Grove, Pa., USA) at 1/10000 dilution in blocking buffer was added (100 μL/well) and incubated at room temperature for one hour. Plates were washed and the bound antibodies were detected using OPD substrate and the reaction stopped adding 4N H₂SO₄. The product was measured at 492 nm in an ELISA plate reader.

### Example 1

### Design of Fc Fusion Protein Derivatives

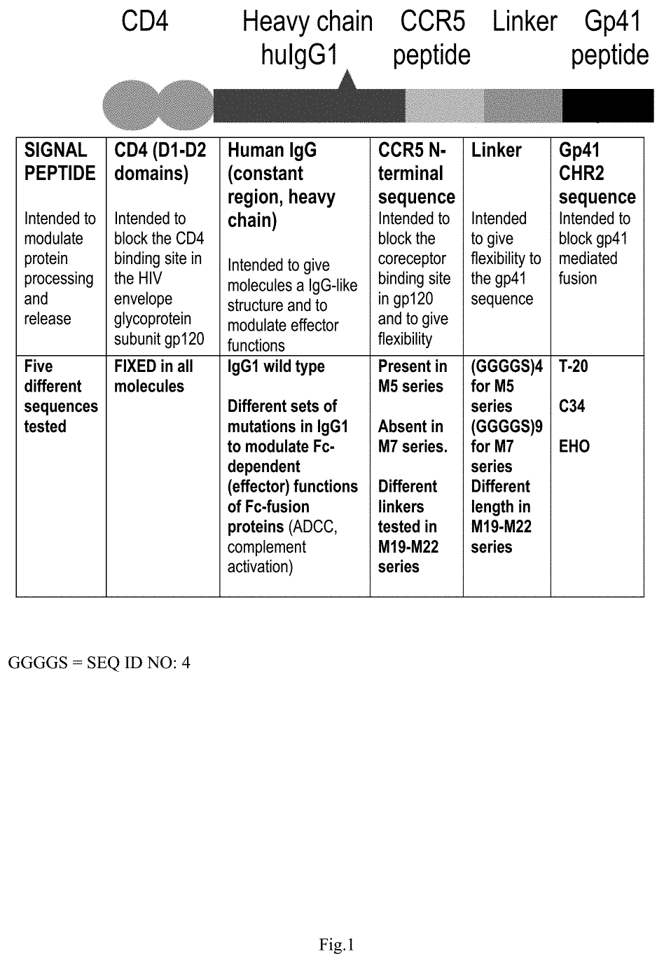

A hu-CD4-murine IgG1 fusion protein was prepared as previously reported. This fusion protein has been used in the past for the identification of anti-CD4 binding site antibodies. See Carrillo J, et al., PLOS One 2015; 10(3):0120648; FIG. 1. However, since CD4-IgG1 molecules are known to have limited therapeutic potential, several changes were introduced to the sequence of the CD4-IgG1 protein to increase its antiviral and ADCC activities. See Jacobson J, et al., Antimicrob Agents Chemother. 2004; 48(2):423-429.

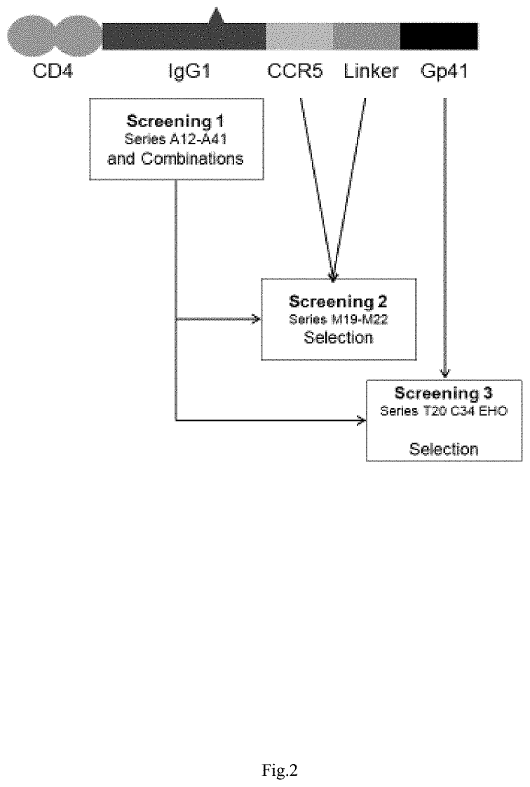

First, the Fc region of human IgG1 was mutated in positions G236A, S239D, A330L and I332E (i.e. AM set of mutations), as described in the art, to increase ADCC mediated responses. See Bournazos, 2014, supra. Further modifications, aimed at increasing the interaction with HIV Env, included the addition of a 29-amino acid sequence corresponding to the N-terminal extracellular region of CCR5 (SEQ ID NO:5) and the addition of a T-20 (SEQ ID NO:10) sequence at the C-terminal end of the Fc chain with a flexible optimal linker. Therefore, a Fc-fusion protein derivative (M5) containing both the CCR5 and T-20 sequences was designed. See FIGS. 1 and 2. Molecules lacking the CCR5 sequence and having an extended linker were also constructed (M7).

Second, several sets of mutations were incorporated into the IgG1 sequence to improve the activity of the M5 and M7 molecules. The sets of mutations tested were as follows: (i) A12 (i.e. F243L, R292P and Y300L; SEQ ID NO:14), (ii) A14 (i.e. F243L, R292P and P396L; SEQ ID NO:15), (iii) A16 (i.e. F243L, R292P, Y300L and P396L; SEQ ID NO:16), (iv) A18 (i.e. F243L, R292P, Y300L, P396L and V305I; SEQ ID NO:17), (v) A41 (i.e. S298A, E333A and K334A; SEQ ID NO:18), (vi) LL (M428L and N434S; SEQ ID NO:19) and combinations thereof (e.g. SEQ ID NO:20-33). See FIGS. 1 and 2, Table 1.

For comparison purposes, an eCD4-IgG1 fusion protein (M1) was also prepared as described previously. At present, the eCD4-IgG1 fusion protein is one of the most potent anti-HIV engineered antibody known in the art. See Gardner, 2015, supra. In order to provide a better basis for comparison, the Fc chain of a CD4-IgG1 fusion protein was further mutated in positions G236A, S239D, A330L and I332E to yield molecule M6. An additional control molecule was prepared by replacing the IgG1 sequence of M7IgG1C34 molecule by the human IgG2 sequence. See FIG. 2. IgG2 lacks ADCC capacity and was, therefore, used as negative control in ADCC analysis.

TABLE 1

| | | Identification |
|---|---|---|
| Molecule set | Mutations | number |
| A | Human IgG1 wt | SEQ ID NO: 3 |
| A-LL | M428L, N434S | SEQ ID NO: 19 |
| AM | G236A, S239D, A330L, I332E | SEQ ID NO: 13 |
| AM-LL | G236A, S239D, A330L, I332E, | SEQ ID NO: 20 |
| | M428L, N434S | |
| A12 | F243L, R292P, Y300L | SEQ ID NO: 14 |
| A12-LL | F243L, R292P, Y300L, M428L, | SEQ ID NO: 21 |
| | N434S | |
| A14 | F243L, R292P, P396L | SEQ ID NO: 15 |
| A14-LL | F243L, R292P, P396L, M428L, | SEQ ID NO: 22 |
| | N434S | |
| A16 | F243L, R292P, Y300L, P396L | SEQ ID NO: 16 |
| A16-LL | F243L, R292P, Y300L, P396L, | SEQ ID NO: 23 |
| | M428L, N434S | |
| A18 | F243L, R292P, Y300L, P396L, | SEQ ID NO: 17 |
| | V305I | |
| A18-LL | F243L, R292P, Y300L, P396L, | SEQ ID NO: 24 |
| | V305I, M428L, N434S | |
| A41 | S298A, E333A, K334A | SEQ ID NO: 18 |
| A41-LL | S298A, E333A, K334A, M428L, | SEQ ID NO: 25 |
| | N434S | |
| A16_1-LL | S239D, F243L, R292P, Y300L, | SEQ ID NO: 26 |
| | P396L, M428L, N434S | |
| A16_2-LL | F243L, R292P, Y300L, K322A, | SEQ ID NO: 27 |
| | P396L, M428L, N434S | |
| A16_3-LL | F243L, R292P, Y300L, I332E, | SEQ ID NO: 28 |
| | P396L, M428L, N434S | |
| A16_4-LL | F243L, R292P, Y300L, K322A, | SEQ ID NO: 29 |
| | I332E, P396L, M428L, N434S | |
| A16_5-LL | F243L, R292P, Y300L, E333A, | SEQ ID NO: 30 |
| | K334A, P396L, M428L, N434S | |
| A16_6-LL | S239D, F243L, R292P, Y300L, | SEQ ID NO: 31 |
| | K322A, I332E, K334A, P396L, | |
| | M428L, N434S | |
| A41_1-LL | S298A, E333A, K334A, F243L, | SEQ ID NO: 32 |
| | R292P, M428L, N434S | |
| A41_A16-LL | F243L, R292P, S298A, E333A, | SEQ ID NO: 33 |
| | K334A, M428L, N434S | |

### Example 2

### Production of Fc Fusion Protein Derivatives

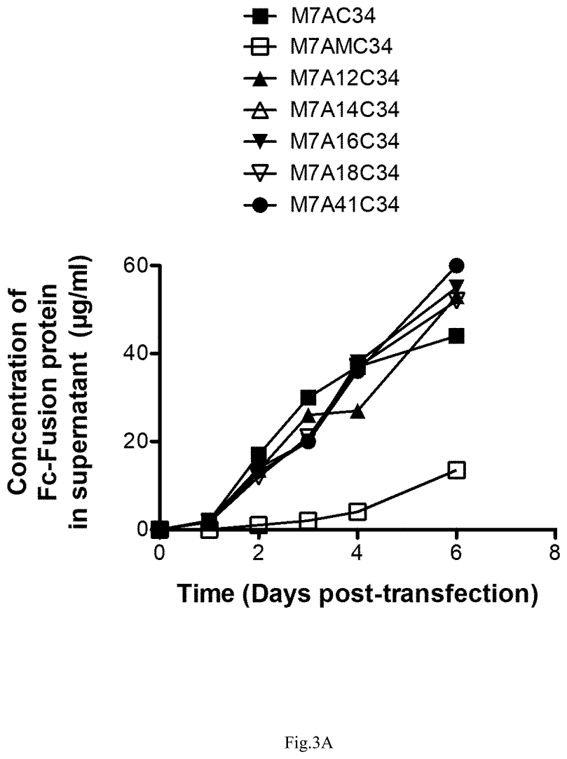

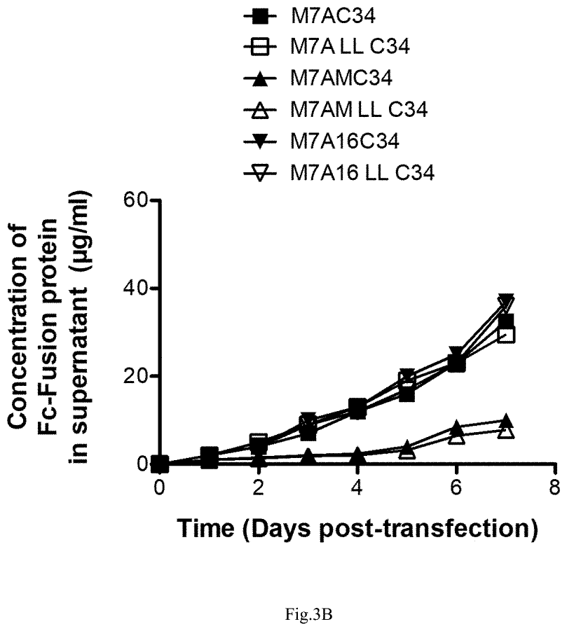

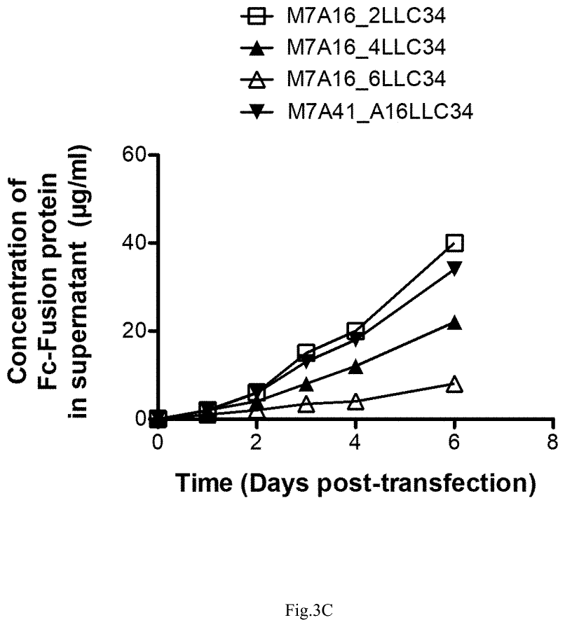

A standard transfection assay was conducted to evaluate the yield of different molecules in mammalian cells (e.g. HEK293 cell line). The level of Fc-fusion proteins released to the cell culture supernatant was analyzed by ELISA, as described above, and the time course of production was determined post-transfection over a period of 6 days. To evaluate the effect of mutations in the IgG1 sequence a set of molecules based on the M7IgG1C34 derivative were analyzed. The molecules included a wild-type IgG1 and several different sets of mutations. See Table 1. While the wild-type IgG1 derivative was produced to high tield, the AM variant showed a c153uclideaparied yield (i.e. roughly 90% reduction). In contrast, all mutants comprising F243L and R292P point mutations (i.e. A12, A14, A16 and A18) and the mutant series A41 showed production yields comparable to IgG1 wild-type molecule. See FIG. 3A. Introduction of LL mutations did not significantly modify the yield of molecules. See FIG. 3B. In contrast, the combination of mutations impacted yield. Indeed, the addition of mutations S239D and I332E (contained in the AM series) to the A16 background had deleterious effects on production. See FIG. 3C.

### Example 3

### Neutralizing Capacity of Fc Fusion Protein Derivatives

A standard neutralization assay was conducted for evaluating the effects of the A12, A14, A16, A18 and A41 sets of mutations over the neutralization capacity of the Fc-fusion protein derivatives of the invention. Four different viruses were employed for the analysis: (i) a HIV-1 strain NL4-3, (ii) a X4-monotropic laboratory isolate, (iii) a HIV-1 strain BaL, a R5-monotropic laboratory isolate and (iv) two HIV-1 primary isolates (i.e. AC10 and SVPB16), both tier 2-type viruses which are particularly difficult to neutralize.

Briefly, in a 96-well culture plate, 100 μL of previously diluted plasma samples were preincubated with 50 μL of pseudovirus stock, using 200 TCID₅₀ at 37° C. for one hour. Then, 100 μL containing 10,000 TZM-bl luciferase-reporter target cells per well were added. Plates were cultured at 37° C. and 5% CO₂ for 48 hours. Serial dilutions of the Fc-fusion protein derivatives were tested, from 1000 to 0.1 ng/mL. TZM-bl reporter cells were treated with dextran (Sigma-Aldrich, Saint Louis, Mo., USA) to enhance infectivity. A luciferase substrate, Britelite Plus (PerkinElmer, Inc., Waltham, Mass., USA), was used for the readout. Non-linear fit of neutralization data was calculated using normalized values fitted to a one site inhibition curve with variable Hill slope to generate IC₅₀ values for each molecule against each virus. All statistical analyses and non-linear fitting were performed using the GraphPad Prism v5.0 software.

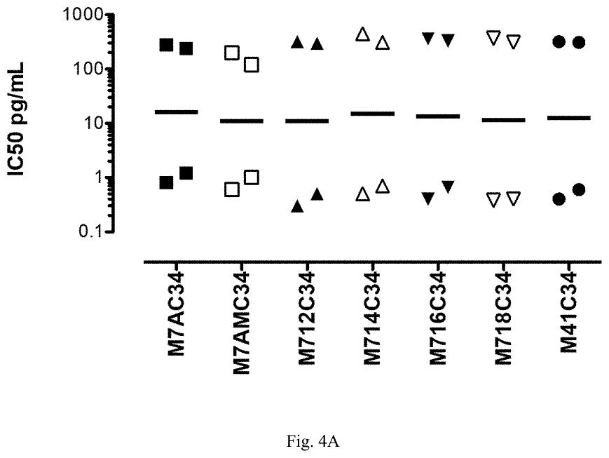

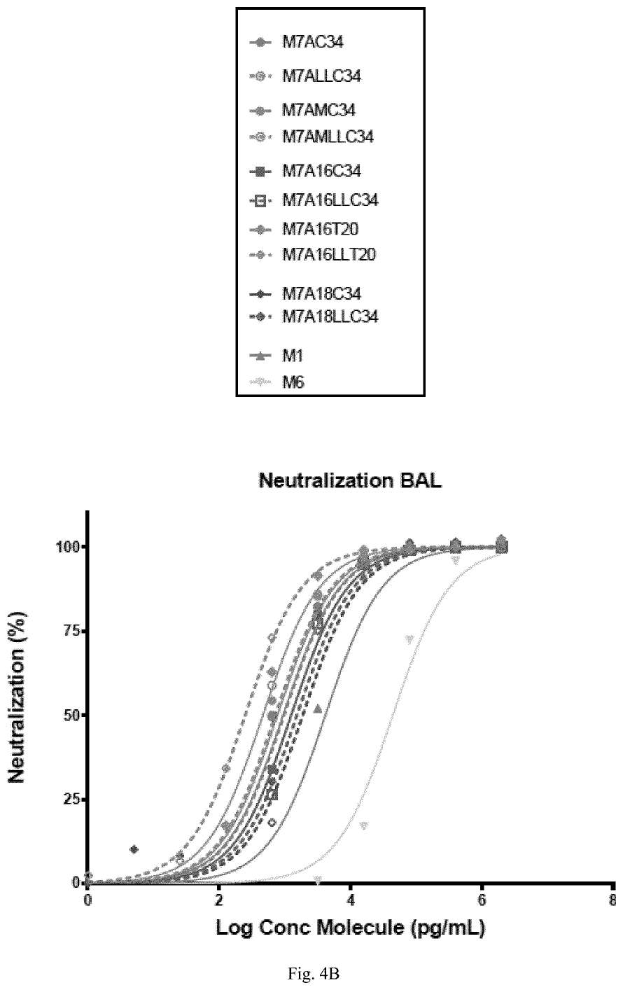

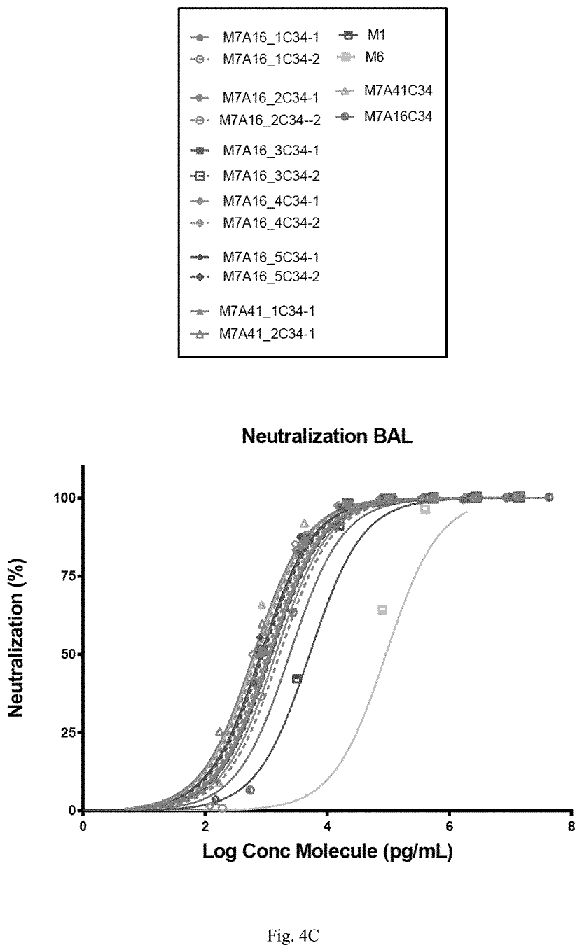

All molecules tested including the wild-type IgG1 and the Fc-fusion protein derivatives containing the AM, A12, A14, A16, A18 and A41 sets of mutations showed similar potency to block HIV infectivity. No significant differences in the geometric means of the IC₅₀ values were observed. See FIG. 4A. The introduction of LL mutations and the combination of different mutations did not significantly modify the neutralizing activity of molecules against the HIV-1 isolate BaL. See FIGS. 4B and 4C.

### Example 4

### Antibody-Dependent Cellular Cytotoxicity (ADCC) Activity of Fc Fusion Protein Derivatives

One of the most relevant features of the Fc-fusion protein derivatives of the invention is their ability to activate NK cells and mediate ADCC. This mechanism is important for the efficient and quick removal of HIV-infected cells and contributes to the protection of uninfected subjects to HIV exposure and acquisition. See Euler Z, et al., AIDS Res Hum Retroviruses 2015; 31(1):13-24.

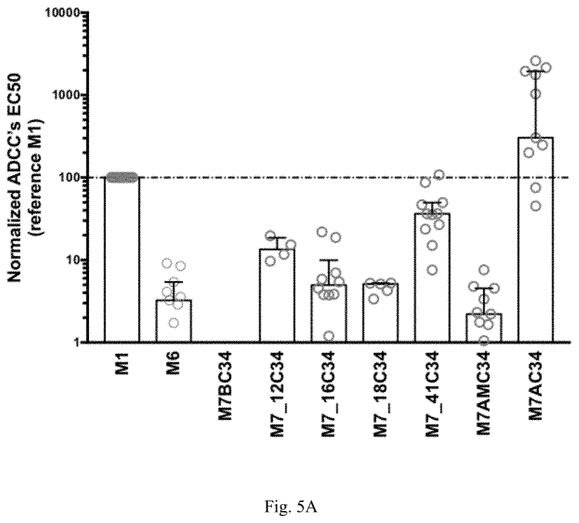

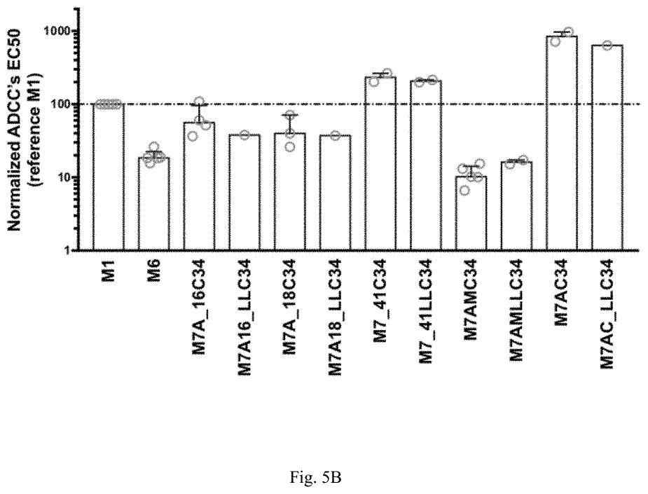

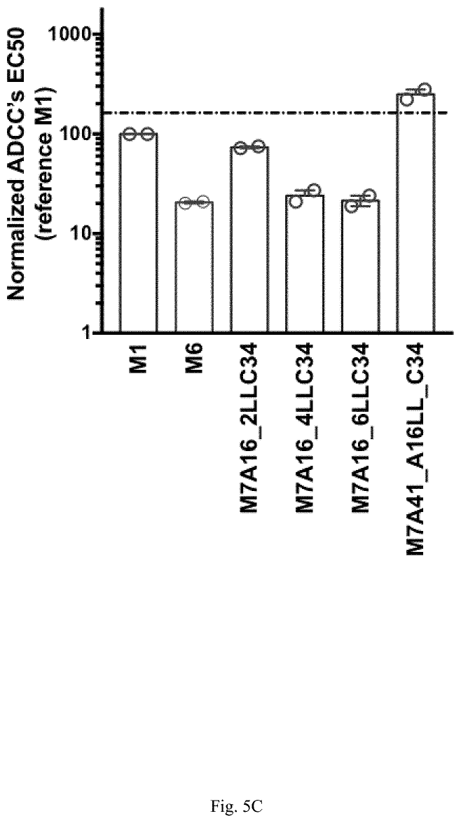

The ADCC activity of the Fc-fusion protein derivatives of the invention was evaluated using a test previously reported in the art. See Alpert M, et al., J. Virol. 2012, 86:12039-12052. In this assay, the killing capacity of NK cells is measured by the reduction of luciferase expression in a reporter cell line infected with HIV. The analysis of dose response curves against cells infected with the HIV BaL isolate was performed by normalizing IC₅₀ values to M1. In a manner consistent with the binding data obtained in ELISA assays, molecules bearing the AM set of mutations (e.g. M6 and M7AMC34) destroyed HIV-infected cells with the lowest IC₅₀ values. See FIG. 5A. On the other hand, molecules bearing the A16 and A18 sets of mutations showed similar IC₅₀ values, while a lower activity was observed in molecules bearing the A41 set of mutations and the IgG1 wild-type molecules (e.g. M7A41C34 and M1). See FIG. 5A. The introduction of LL mutations did not significantly modify the ADCC activity of molecules. See FIG. 5B. In contrast, the combination of mutations improved in some cases the ADCC activity. Indeed, combinations A16_4 LL and A6_6 LL showed the highest activity, which was comparable with the M6 reference molecule. See FIG. 5C.

### Example 5

### Binding Affinity of Fc Fusion Protein Derivatives to Human Fc Receptors and C1q

The binding affinities of the Fc-fusion protein derivatives of the invention to different Fc receptors were assayed. These molecules were designed with the specific purpose of increasing their binding affinity to human CD16. However, the sets of mutations introduced could also affect the binding affinities of the Fc-fusion protein derivatives of the invention to other Fc receptors and provoke unedesirable side-effects. Among human Fc receptors, CD64, CD32 and CD16 are the most relevant to mediate effector functions in antibodies and Fc-fusion proteins. See Vogelpoel L, et al., Front. Immunol. 2015; 6(79):1-11. Therefore, the binding affinities of several Fc-fusion protein derivatives of the invention to recombinant human CD64, CD32a, CD32b/c and CD16 (both V and F isoforms) were assayed by ELISA.

The binding affinities of the Fc-fusion protein derivatives of the invention to C1q was assayed to assess potential complement activation. Therefore, the binding affinities of several Fc-fusion protein derivatives of the invention to recombinant human C1q, a complement component, was assayed by ELISA.

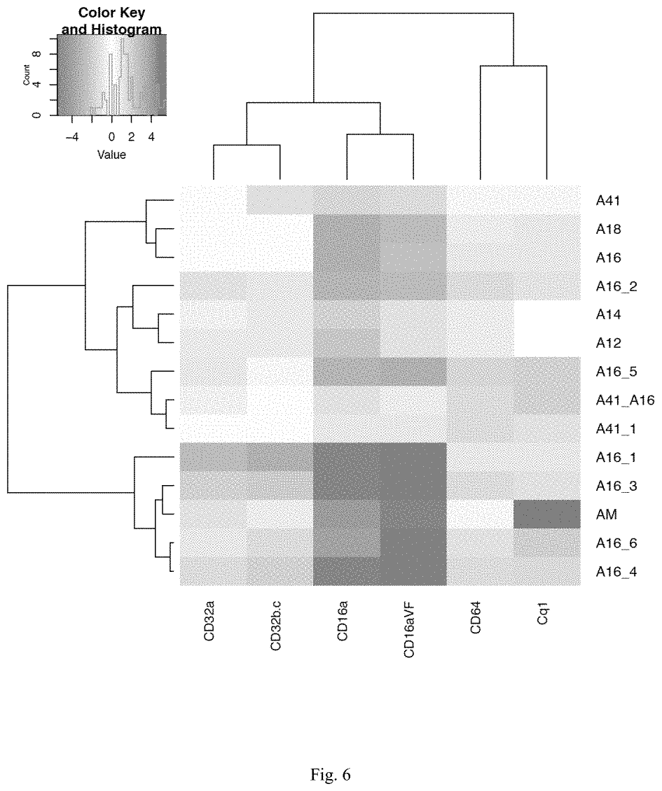

All ELISA data were normalized to the wild type molecule M7AC34 containing a unmutated human IgG1. Signal ratios were Log2 transformed. Molecules and receptors were clustered 56uclideanlidean distance similarity after data normalization. See FIG. 6. This global analysis identified minimal and homogeneous changes in the affinity of the different molecules for CD64. Regarding CD32a and CD32b/c, minimal changes occurred except for molecules A16_1, which showest the highest affinity. In contrast, some molecules displayed low or null affinity for C1q (molecuels A16_4 and AM, respectively). See FIG. 6. All molecules increased affinity for CD16 to different extent, being the most potent derivatives grouped in a cluster encompassing molecules A16_1, A16_3, Am, A16_6 and A16_4.

### Example 6

### Combination of Mutations Enhancing ADCC and Extending Activity

The Fc section of molecules containing the wild-type IgG1 sequence and the AM and A16 sets of mutations were further modified to include the LL set of mutations (i.e. M428L and N434S). The LL set of mutations has been related to extended activity of recombinant antibodies in human plasma. See Roopenian D, et al., Nature Reviews Immunology. 2007; 7:715-725.

First, the different combinations of mutations were tested in transfection assays to evaluate their potential effect over production yields. The addition of mutations M428L and N434S to the IgG1 wild-type M7AC34 molecule, or the M7AMC34 and M7A16C34 molecules, did not affect their production yields, neutralizing and ADCC activity. See FIGS. 3-5.

The results above suggest that the introduced LL set of mutations did not impact negatively the HIV antiviral and ADCC activities of the Fc-fusion protein derivatives of the invention.

### Example 7

### Effect of LL Set of Mutations on the Binding Affinity of Fc Fusion Protein Derivatives to Human Neonatal Fc (FcRn) Receptors

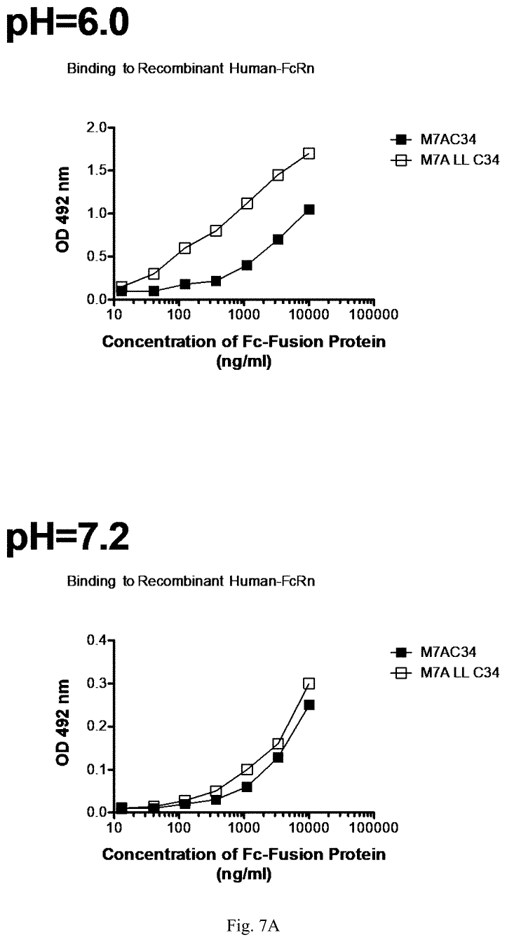

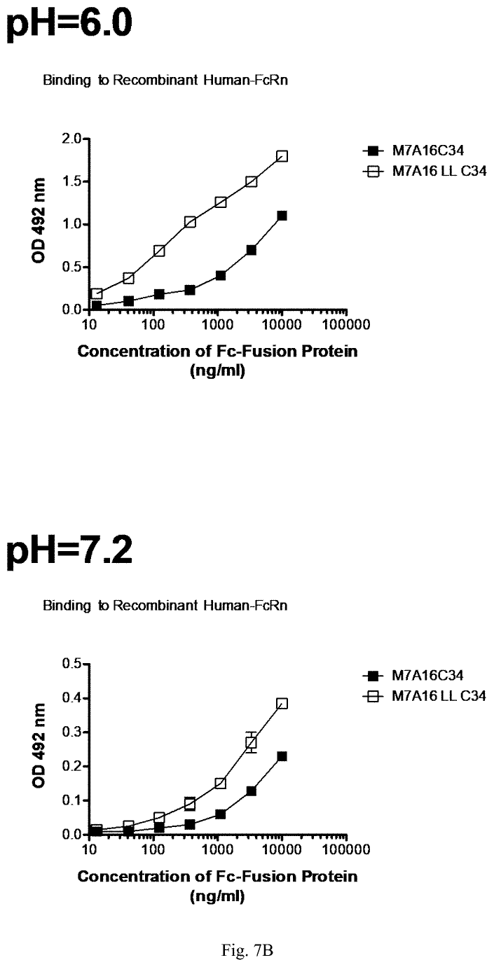

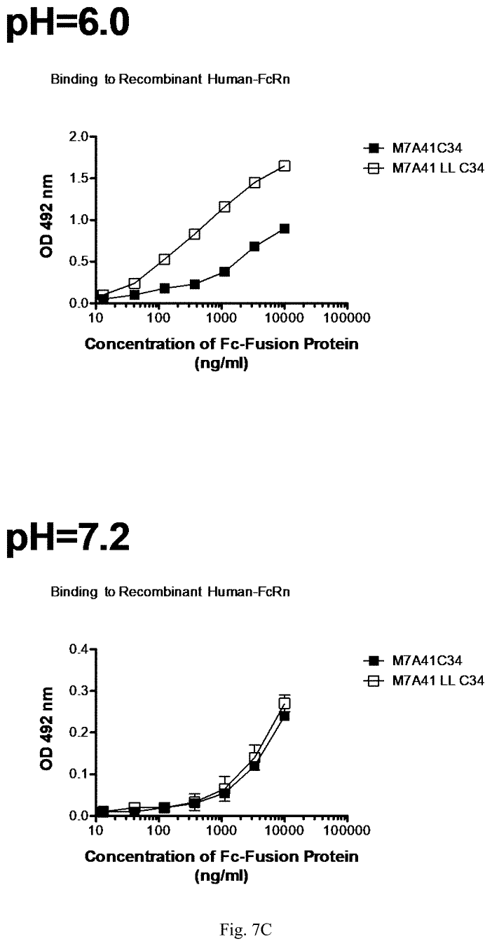

ELISA tests were performed for assaying the changes in affinity for FcRn receptors induced by the LL set of mutations in the Fc-fusion protein derivatives of the invention. The assays were conducted using two different pH conditions: pH=6.0 and pH=7.2. All molecules bearing mutations the LL set of mutations (M7ALLC34, M7A16LLC34 and M7A41LLC34) showed significantly higher affinity for FcRn receptors than their unmutated counterparts (M7AC34, M7A16C34 and M7A41C34) at pH 6.0 (mean increase 10×), while the effect was much lower at pH=7.2. See FIGS. 7A-7C. In summary, these data suggest that the LL set of mutations maintain both the neutralizing and ADCC activities of the Fc-fusion protein derivatives of the invention, while extendig their activity.

### Example 8

### Analysis of the Effects of Linker Sequences on Protein Production and Activity

Although the M7A16_4 LLC34 molecule showed a convenient profile of production, neutralization and ADCC activities, the Fc-fusion protein derivatives were further modified to include the CCR5 (SEQ ID NO:5) sequence and different linkers with different flexibility. To this end, a M7A16T20 molecule including the T20 (SEQ ID NO:10) or the C34 sequence (SEQ ID NO:11) was constructed based on the M7A16_4 LLT20 molecule. Similarly, linker peptides M19, M20, M21 and M22 (SEQ ID NO:6-9 were also tested generating molecules M19A16_4 LLT20, M20A16_4 LLT20, M21A16_4 LLT20 and M22A16_4 LLT20, and their respective C34 derivatives.

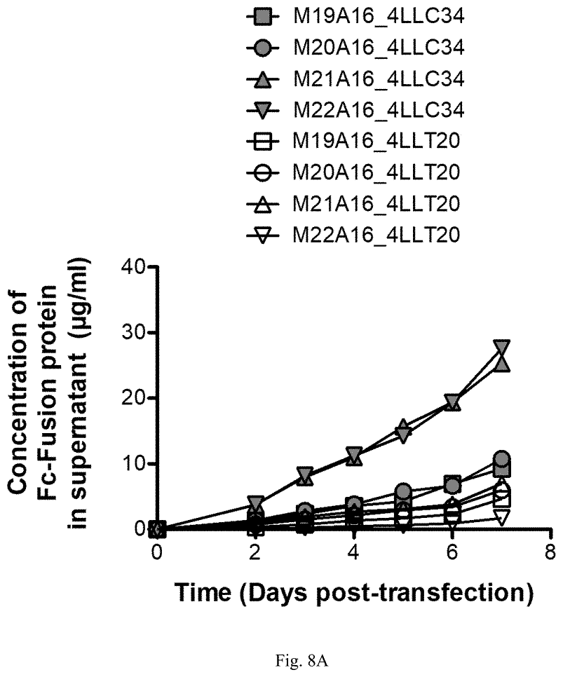

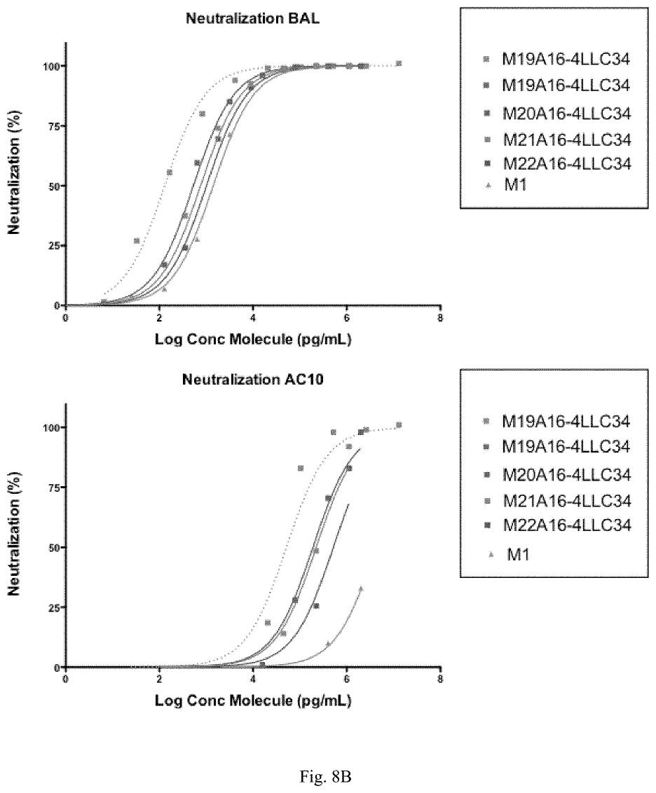

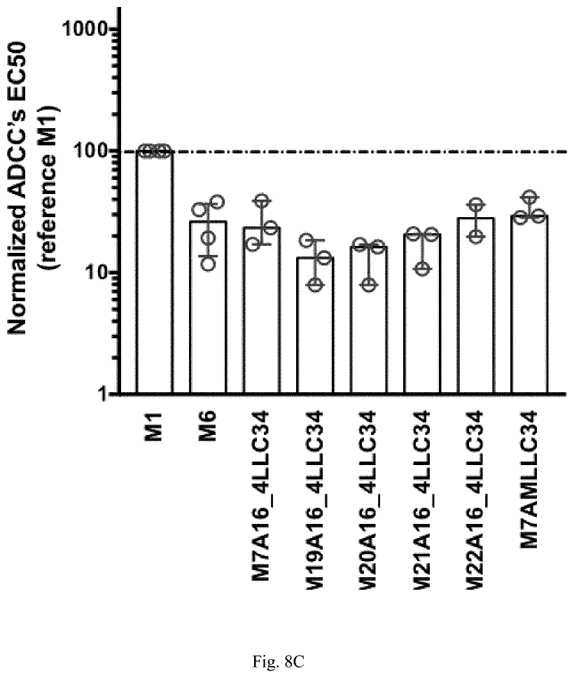

In terms of production, all T20 derivatives showed poorer yield than the C34 counterparts. The different linkers also impacted production, as molecules bearing the M21 and M22 linkers showed the highest yield. See FIG. 8A. Furthermore, although neutralization capacity was roughly similar for all molecules; M22 derivatives shwed consistently lower potency at neutralizing the HIV-1 isolates BaL or AC10. See FIG. 8B.

In terms of ADCC activity, a similar rank was observed, being the M22 derivative less potent than their M19-M21 conunterparts. Surprisingly M19, M20 and M21 derivatives showed highest activity than the reference molecule M6. See FIG. 8C.

### Example 9

### Analysis of the Effects of Gp41 Sequences on Protein Production and Activity

To improve yield and activity profile, the Fc-fusion protein derivatives were further modified to include HIV-2 peptide EHO (SEQ ID NO:12) in the C-terminal end. Derivatives M19A16_4 LLEHO, M20A16_4 LLEHO and M21A16_4 LLEHO were constructed and compared to their C34 counterparts.

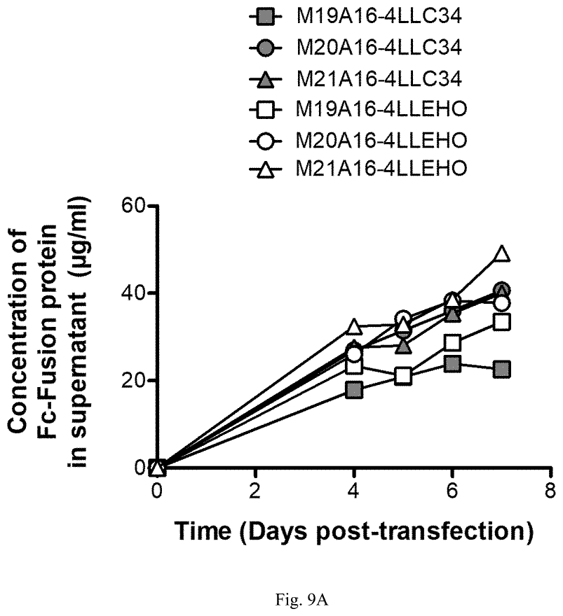

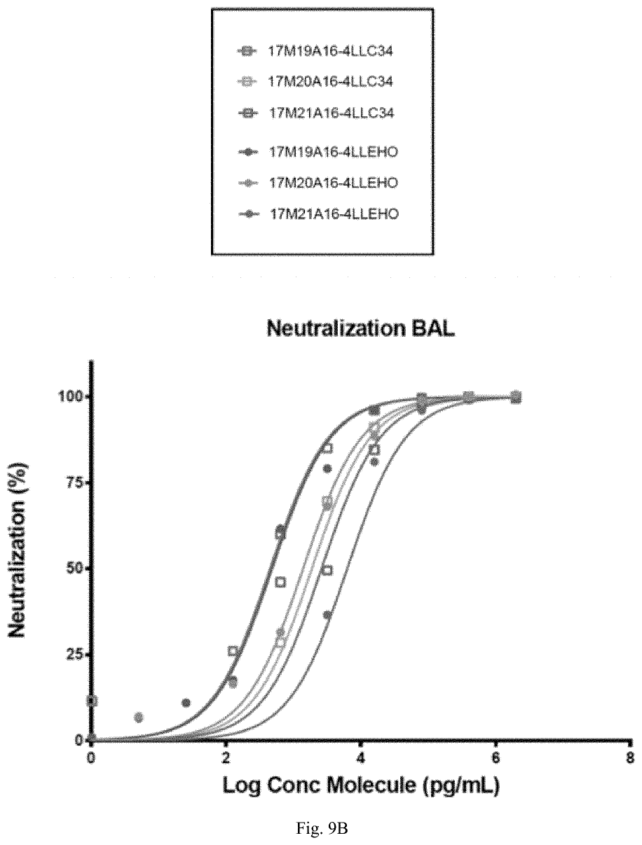

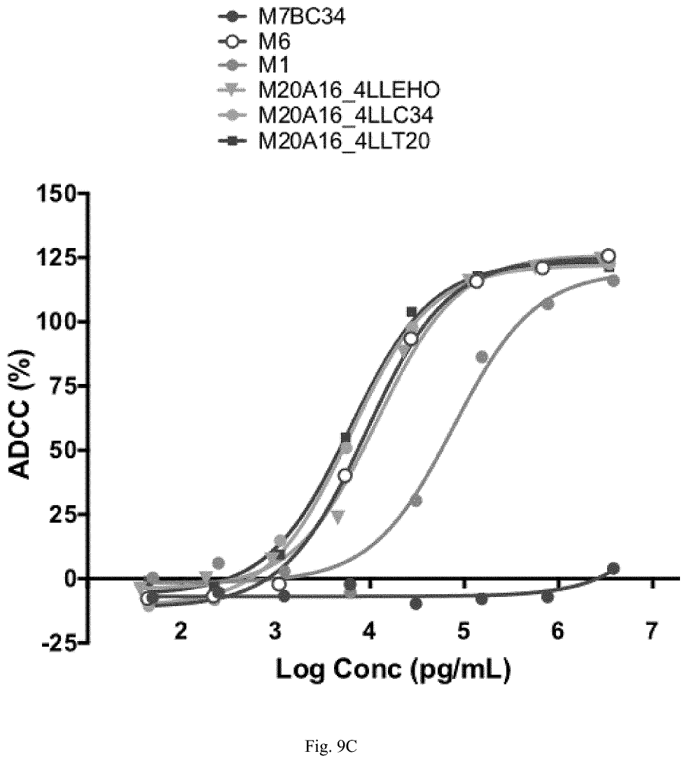

In terms of production, molecules bearing the EHO peptide showed the highest yield, reaching production rates comparable to the WT M7AC34 molecule. See FIG. 9A. Remarkably this gain in yield is accompanied by a high neutralization and ADCC potencies See FIG. 9B and FIG. 9C.

### Example 10

### Viral Inactivation Activity of Fc Fusion Protein Derivatives

To evaluate the irreversible inactivation of HIV induced by the Fc-fusion protein derivatives of the invention, a highly infectious HIV BaL viral stock was incubated in DMEM culture medium with serial dilutions (concentration range: 0.7 μg/mL to 0.7 pg/mL) of Fc-fusion protein derivatives of the invention (concentration range: 1 μg/mL to 1 pg/mL). After 1 hour of incubation at 37° C., samples were diluted with DMEM and viruses and soluble proteins were separated by the addition of LentiX Concentrator reagent (Takara/Clontech Laboratories, Inc., Mountain View, Calif., USA). The mixture was incubated for 30 min at 4° C. and was subsequently centrifuged at 1,500×g for 45 minutes at 4° C. Supernatants were removed and viral pellets were re-suspended in DMEM. The infectivity of the treated viruses was analyzed by titration in TZM-bl cells. See Li M, et al., J. Virol. 2005; 79:10108-10125. The TCID₅₀ value was calculated for each preparation to assess the remaining infectivity of treated viruses.

### Example 11

### AAV Mediated Expression of Fc Fusion Protein Derivatives In Vivo

NSG mice (Jackson Laboratory, Bar Harbor, Me., USA) were maintained by brother-sister mating under specific pathogen-free (SPF) conditions.

To induce stable expression of Fc-fusion protein derivatives in these animals, their sequences were cloned into AAV8 expressing plasmids (CBATEG, Universitat Autonoma de Barcelona, Barcelona, ES). 1×10¹¹ viral particles were diluted into 40 μL of 100 mM sodium citrate, 10 mM Tris, pH 8 buffer and injected into the gastrocnemius muscle of eight weeks old mice. At the same time, mice were humanized by intraperitoneal injection of 10 million of PBMCs isolated from healthy individuals. After two weeks, mice were infected by intraperitoneal injection of 10000 TCDI₅₀ of NL4-3 HIV viral isolate. Blood samples were collected weekly and the CD4+ and CD8+ T cells count was analyzed by flow cytometry using Perfect Count beads (Cytognos SL, Salamanca ES) in combination with the following antibodies: anti-human CD45-V450, CD3-APC/Cγ7, CD4-APC, CD8-V500, CD14-PerCP/Cγ5.5, CD56-PE, CD16-Fitc and anti-mouse CD45 PE/Cγ7. Samples were analyzed using a LSR II flow cytometer (BD Biosciences Corp., Franklin Lakes, N.J., USA). Samples were also assayed for Fc-fusion protein derivative levels using the above described ELISA approach. After 3 weeks from infection, mice were sacrificed and blood and tissue samples were collected. Samples were analyzed by flow cytometry and viral load and total HIV DNA were determined by qPCR.

### Example 12

### Passive Immunization Protocol with Plasmidic Vectors

NSG mice (Jackson Laboratory, Bar Harbor, Me., USA) were maintained by brother-sister mating under specific pathogen-free (SPF) conditions.

The plasmids pM5A16T20 and pM7A16LLC34 were produced in endotoxin free conditions using EndoFree Plasmid Kits (Qiagen NV, Venlo, NL). For transient expression of Fc-fusion protein derivatives in vivo, plasmids were administered to NGS mice by intramuscular or intravenous injections. After plasmid administration, blood samples were collected weekly and were assayed for Fc-fusion protein derivative levels using the above described ELISA approach. After 4 weeks from plasmid administration, mice were sacrificed and blood and tissue samples were collected and analyzed for Fc-fusion protein derivative levels and for anti-idiotype antibodies.

### Example 13

### In Vivo Activity

Fc-fusion protein derivatives were produced in large volume culture of HEK-293T cells by transient transfection. The recombinant protein was purified after loading supernatants in CaptureSelect FcXL Affinity Matrix (Thermo Fisher Scientific, Waltham, Mass., USA) columns and eluting bound protein with glycine buffer pH=3.5. After pH neutralization, dialysis and concentration, a highly pure stock of 5 mg/mL was obtained.

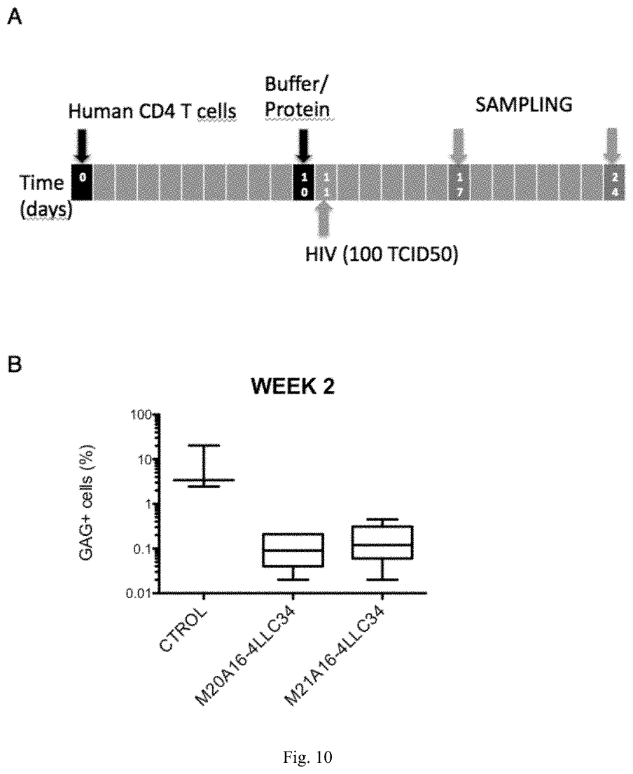

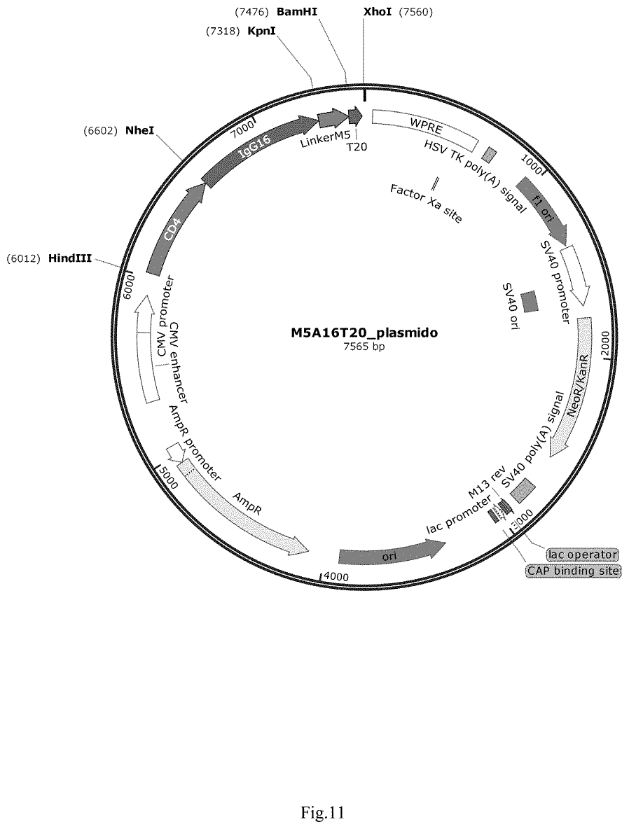

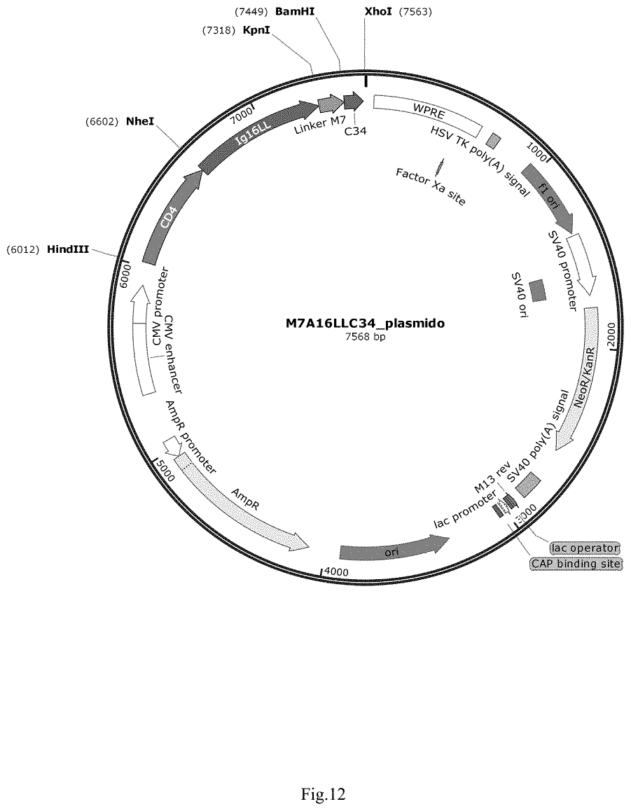

NSG immunodeficient mice were humanized by intraperitoneal injection of 10 million of PBMCs isolated from healthy individuals. After two weeks, mice were infected by intravenous injection of 100 TCID₅₀ of NL4-3 HIV viral isolate. Mice were treated 24 hours before infection with 1 mg of purified Fc-fusion protein derivative in 200 μL or with the same volume of PBS by intraperitoneal injection. Blood samples were collected at week one and two after infection by maxillary vein puncture and processed to obtain plasma samples See FIG. 10A. Plasma samples were assayed for the levels of viremia using the Abbott Real Time HIV-1 (Abbott Laboratories, Abbott Park, Ill., USA) while blood cells and spleenocytes were assayed for productive infection by assessing the frequency og HIV-1 GAG+ cells. Both M20A16_4 LLC34 and M21A16_4 LLC34 molecules maintained the frequency of infected cells below the limit of detection after two weeks of infection. In contrast, control group showed a median of 3.3% of GAG positive cells See FIG. 10B.

### Example 14

### Detection of HIV Env Glycoproteins

To assess the ability of the Fc-fusion protein derivatives of the invention to identify HIV proteins in sample, MOLT cells chronically infected with the HIV isolates NL4-3 or BaL were incubated with increasing amounts of Fc-fusion protein derivatives. Molecules bound to these cells were revealed with a mouse anti human IgG antibody coupled with the fluorochrome Phycoerythrine (PE/Jackson ImmunoResearch Laboratories, Inc., West Grove, Pa., USA) and analyzed by flow cytometry in a LSRII flow cytometer (BD Biosciences Corp., Franklin Lakes, N.J., USA).

What is claimed is:

1. An Fc-fusion protein derivative comprising from the N- to the C-terminus:
(a) the D1 and D2 extracellular domains of a human CD4,
(b) the Fc portion of a human IgG1 comprising at least one of a M428L or N434S point mutations,
(c) a moiety selected from the group consisting of (i) a linker polypeptide of sequence (GGGGS)ₙ wherein 1≤n≤10, (ii) SEQ ID NO:5-9 and (iii) combinations thereof and
(d) a gp41-derived polypeptide.

1. The Fc-fusion protein derivative according to claim 1, wherein the Fc portion of the human IgG1 comprises at least one of a G236A, S239D, A330L or I332E point mutations.

2. The Fc-fusion protein derivative according to claim 1, wherein the Fc portion of the human IgG1 further comprises a F243L point mutation.

3. The Fc-fusion protein derivative according to claim 1, wherein the Fc portion of the human IgG1 further comprises a R292P point mutation.

4. The Fc-fusion protein derivative according to claim 1, wherein the Fc portion of the human IgG1 further comprises a Y300L point mutation.

5. The Fc-fusion protein derivative according to claim 1, wherein the Fc portion of the human IgG1 further comprises a P396L point mutation.

6. The Fc-fusion protein derivative according to claim 1, wherein the Fc portion of the human IgG1 further comprises at least one of a S298A, E333A or K334A point mutations.

7. The Fc-fusion protein derivative according to claim 1, wherein the Fc portion of the human IgG1 further comprises a K322A or V305I point mutation.

8. The Fc-fusion protein derivative according to claim 1, wherein the human CCR5 receptor sequence comprises SEQ ID NO:5.

9. The Fc-fusion protein derivative according to claim 1, wherein the gp41-derived polypeptide comprises a T-20, C34 or EHO polypeptide.

10. An isolated nucleic acid encoding the Fc-fusion protein derivative according to claim 1.

11. The nucleic acid according to claim 11, wherein the nucleic acid is codon optimized.

12. An expression vector comprising the nucleic acid according to claim 12.

13. A host cell comprising the Fc-fusion protein derivative according to claim 1.

14. A pharmaceutical composition comprising a therapeutically effective amount of the Fc-fusion protein derivative according to claim 1.

15. A method of treating HIV infection or AIDS in a subject in need thereof, the method comprising:
administering the Fc-fusion protein derivative according to claim 1 to the subject.

16. A method of lowering likelihood of HIV infection or AIDS in a subject in need thereof, the method comprising:
administering the Fc-fusion protein derivative according to claim 1.

17. A combination comprising the Fc-fusion protein derivative according to claim 1, and at least one therapeutic agent.

18. The combination according to claim 18, wherein the at least one therapeutic agent is an HIV antiretroviral.

19. A method for preparing the Fc-fusion protein derivative according to claim 1, the method comprising:
(a) culturing a host cell comprising an isolated nucleic acid encoding the Fc-fusion protein derivative,
(b) expressing the nucleic acid, and
(c) recovering the Fc-fusion protein derivative encoded by the amino acid from the host cell culture.

20. A method of inactivating HIV, the method comprising contacting the virus with a Fc-fusion protein derivative according to claim 1.

21. A method of inducing the expression of gp120 in a HIV infected cell, comprising contacting the infected cell with the Fc-fusion protein derivative according to claim 1.

22. A method of detecting HIV in a sample comprising:
(a) contacting the sample with a Fc-fusion protein derivative according to claim 1; and
(b) determining whether the Fc-fusion protein derivative specifically binds to a molecule of HIV in the sample.
